SEQUENCE LISTING (8 sequences)

SEQ ID NO: 1 - DNA, 20 bp, Artificial
  Primer sequence
ttattcccacctgccgctaa

SEQ ID NO: 2 - DNA, 21 bp, Artificial
  Primer
ctgccccgaacttcttctctt

SEQ ID NO: 3 - DNA, 22 bp, Artificial
  Probe
cccccaaccttaaatgcctggg

SEQ ID NO: 4 - DNA, 27 bp, Artificial
  Primer
ttggattggcagagagcctgttgggat

SEQ ID NO: 5 - DNA, 23 bp, Artificial
  Primer
tacccaggcatttaaggttcggg

SEQ ID NO: 6 - DNA, 25 bp, Artificial
  Primer
aataccgtctgcgtcatcttttgcc

SEQ ID NO: 7 - DNA, 29 bp, Artificial
  Primer
agcacagcgacatctagcagcggacacag

SEQ ID NO: 8 - DNA, 27 bp, Artificial
  Primer
agccgcctagcttatccagtgcagcaa

(19) United States
(12) Patent Application Publication
(10) Pub. No.: US 2013/0085146 A1
(43) Pub. Date: Apr. 4, 2013

(54) LIPID-DRUG FORMULATIONS AND METHODS FOR TARGETED DELIVERY OF LIPID-DRUG COMPLEXES TO LYMPHOID TISSUES

(71) Applicants: Rodney J.Y. Ho, Seattle, WA (US); Che-Chung Tsai, Seattle, WA (US)

(72) Inventors: Rodney J.Y. Ho, Seattle, WA (US); Che-Chung Tsai, Seattle, WA (US)

(21) Appl. No.: 13/466,345

(22) Filed: May 8, 2012

Related U.S. Application Data
(63) Continuation-in-part of application No. 10/757,775, filed on Jan. 14, 2004.
(60) Provisional application No. 60/440,220, filed on Jan. 14, 2003.

(51) Int. Cl. A61K 47/24; A61K 31/496

(52) U.S. Cl. CPC A61K 47/24; A61K 31/496
USPC 514/253.01; 514/785

(57) ABSTRACT

The present application is directed to lipid-drug complexes and related methods for producing stable lipid-drug complexes at or near the neutral pH range and administering pharmaceutical lipid-drug complexes to patients. In certain examples, the lipid-drug complex has a lipid-to-drug molar ratio of approximately 3:1. In certain examples, the lipid-drug complex may have lipid-to-drug molar ratios of less than 3:1 to 10:1 or higher. The present application is also directed to methods of administering a drug to a patient though subcutaneous injection of the lipid-drug complexes into particular tissues to effect higher localized concentrations of the at least one drug. Lipid-anti-HIV-drug complexes can be subcutaneously injected into the lymphoid tissue of a HIV-infected mammalian subject via the lymphatic vessels to deliver high concentrations of stable lipid-anti-HIV-drug complexes, rather than delivery of the anti-HIV drug intravenously via the blood stream which will eventually reach the lymphatic system at lower concentrations.

### CROSS REFERENCE TO RELATED APPLICATIONS

This application is a continuation-in-part of patent application Ser. No. 10/757,775, filed Jan. 14, 2004, which claims the benefit of Provisional Patent Application No. 60/440,220, filed Jan. 14, 2003.

### TECHNICAL FIELD

The present application is directed to compositions and related methods for the delivery of pharmaceutical agents to the lymphoid system, and in particular, to the lymphoid-specific delivery of various lipid-pharmaceutical and lipid-biological complexes.

### BACKGROUND

Since the emergence of the Human Immunodeficiency Virus/Autoimmune Deficiency Syndrome (“HIV/AIDS”) epidemic in the 1980's, the total number of HIV/AIDS-related death is estimated to be 17.5 million globally. More recent global statistics of the HIV/AIDS epidemic suggest a grim picture. For the year 2003, approximately 5 million people are estimated to have been newly infected with the retrovirus, HIV, involving 4.2 million adults and 700,000 children less than fifteen years of age. The number of people who are living with the HIV/AIDS in 2003 is estimated to be 40 million, of which 37 million are adults and 2.5 million are children. For the year 2003, approximately 3 million HIV/AIDS-related deaths are estimated, which includes 2.5 million adults and 500,000 children. Although the estimates for 2003 are lower than those published for 2002, the number of people living with HIV/AIDS is not decreasing, nor is there a decline in the epidemic. From 1998 to 2002, the estimated number of deaths among persons with AIDS declined 14%. However, AIDS prevalence, or the number of persons living with AIDS, continues to increase. At the end of 2002, an estimated 384, 906 persons in the United States were reported to be living with AIDS. The term HIV/AIDS may refer to three categories of cases: (1) new diagnosis of HIV infection only; (2) new diagnoses of HIV infection with later diagnoses of AIDS; and (3) concurrent diagnoses of HIV infection and AIDS.

In order to treat HIV/AIDS-afflicted patients, an aggressive form of therapy was implemented in 1996, known as the “highly active anti-retroviral therapy” or (“HAART”), in which a plethora of drugs are administered to patients systemically. The clinical urgency for drugs having more potent anti-HIV effects has motivated the development of various types of anti-HIV drugs, including nucleoside analogs (e.g., dideoxynucleoside derivatives, including 3′-azido-3′-deoxythymidine (“AZT”), dideoxy cytidine (“ddC”), and dideoxy inosine (“ddI”), protease inhibitors, and phosphonoacids (e.g., phosphonoformic and phosphonoacetic acids). Many of these anti-HIV agents are lipid-derivatized or incorporated into liposomes prior to systemic administration (Hostetler, Ky. et al., Methods of treating viral infections using antiviral liponucleotides, Ser. No 09/846,398, US 2001/0033862; U.S. Pat. No. 5,223,263; Hostetler, Ky. et al., Lipid derivatives of phosphonoacids for liposomal incorporation and method of use, U.S. Pat. No. 5,194,654; Gagne J. F. et al., Targeted delivery of indinavir to HIV-1 primary reservoirs with immunoliposomes, Biochem Biophys Acta. 1558(2):198-210, [February 2002]). For example, some anti-HIV drugs were encapsulated into the aqueous core of multilamellar and polyethyleneglycerol derivatized liposomes (“PEGylated”) (Bergeron, M G. et al., Targeting of infectious agents bearing host cell proteins, WO 00/66173 A3; Bergeron, M G. et al., Liposomes encapsulating antiviral drugs, U.S. Pat. No. 5,773,027; Bergeron, M G. et al., Liposome formulations for treatment of viral diseases, WO 96/10399 A1; Gagne J F et al., Targeted delivery of indinavir to HIV-1 primary reservoirs with immunoliposomes, Biochem Biophys Acta. 1558(2):198-210, February 2002; Dufresne I et al., Targeting lymph nodes with liposomes bearing anti-HLA-DR Fab' fragments, Biochem Biophys Acta. 1421(2):284-94 [1999]; Bestman-Smith J et al., Sterically stabilized liposomes bearing anti-HLA-DR antibodies for targeting the primary cellular reservoirs of HIV-1 Biochem Biophys. Acta. 1468(1-2):161-74 [2000]; Bestman-Smith J et al., Targeting cell free HIV and virally-infected cells with anti-HLA-DR immunoliposomes containing amphotericin B, AIDS 10;14(16):2457-65 [2000]; Harvie P, Desormeaux A et al., Lymphoid tissues targeting of liposome-encapsulated 2′,3′-dideoxyinosine, AIDS 9(7):701-7 [1995]).

### Development of Lipid-Drug Complexes as a Mechanism for Drug Delivery

In general, lipid-drug complexes are formed from the aggregation of lipid molecules and pharmaceutical agents, in which the lipid component is a major constituent. Lipid-drug complexes are colloidal particles that can adopt certain configurations, such as an enclosed lipid bilayer or a lipid-drug sheet-disk complex. Lipid-drug complexes, including various forms of liposomes or lipid vesicles, can be prepared by employing lipid molecules derived from either natural sources or formed by chemical synthesis. Although lipid constituents can vary, many formulations employ synthetic products consisting of natural phospholipids, mainly phosphatidylcholine. Most of the liposome formulations approved for human use contain a phosphatidylcholine component comprising a neutral head group and fatty acyl chains of variable lengths and degrees of saturation. A fraction of cholesterol (˜30 mol %) can be included in the lipid formulation in order to modulate the rigidity and to reduce the serum-induced instability caused by the binding of serum proteins to the liposome membrane.

The composition of the lipid head group and the pH of the operative environment determine whether the liposomes formed bear a negative, neutral, or positive charge on the liposome surfaces. The nature and the density of charge on the surface of liposomes influence the stability, the kinetics, and the extent of biodistribution, as well as the interaction with and uptake of liposomes by target cells. Liposomes with a neutral surface charge have a lower tendency to be cleared by cells of the reticuloendothelial system (“RES”) after systemic administration and have the highest tendency to aggregate. Although negatively charged liposomes are less likely to aggregate and are more stable in suspensions relative to neutral liposomes, negatively charged liposomes are prone to nonspecific-cellular uptake in vivo. Negatively charged liposomes containing phosphatidylserine (“PS”) or phosphatidylglycerol (“PG”) were observed to be endocytosed at a faster rate and to a greater extent than neutral liposomes (Allen T M, et al., Liposomes containing synthetic lipid derivatives of polyethylene glycol) show prolonged circulation half-lives in vivo, Biochim Biophys Acta 1066:29-36 [1991]; Lee R. J, et al., Folate-mediated tumor cell targeting of liposome-entrapped doxorubicin in vitro, Biochem Biophys Acta 1233:134-144 [1995]). Presumably, the negative surface charge is recognized by receptors found on a variety of cells, including macrophages (Allen T M et al. [1991]; Lee R J, et al., Delivery of liposomes into cultured KB cells via folate receptor-mediated endocytosis, J Biol Chem 269:3198-3204 [1994]).

The inclusion of some glycolipids, such as the ganglioside GM₁ or phosphotidylinositol (“PI”), inhibits the liposome uptake by macrophages and RES cells, and prolongs the duration of liposome circulation. A small amount of negatively charged lipids can stabilize neutral liposomes against an aggregation-dependent uptake mechanism (Drummond D C, et al., Optimizing liposomes for delivery of chemotherapeutic agents to solid tumors, Pharmacol Rev 51:691-743 [1999]). Positively charged, cationic liposomes, often used as a DNA condensation reagent for intracellular DNA delivery in gene therapy, interact with serum proteins. The aggregates of liposome and serum proteins are recognized by RES cells, and the uptake by RES cells promotes clearance in the lung, liver, and spleen. This mechanism of RES-mediated clearance partly explains the low levels of in vivo transfection efficiency. Other factors such as DNA instability, immune-mediated clearance, inflammatory response, and non-accessibility to target tissue can also contribute to low transfection efficiency levels in mammals. High doses of positively charged liposomes can produce varying degrees of tissue inflammation (Scheule R K, et al., Basis of pulmonary toxicity associated with cationic lipid-mediated gene transfer to the mammalian lung, Hum. Gene. Ther 8:689-707 [1997]).

Although the technology for forming primitive forms of hydrated lipid bilayer films as spherical vesicles or liposomes was developed in the 1960's, the potential for applying lipid-drug complexes as a drug-delivery system was not realized until 30 years later. In general, the lipid component of a lipid-drug complex can be modified to exhibit biodegradable or biocompatible properties so that various lipid-drug complexes can be produced to function as an ideal drug carrier. Typically, a lipid-drug complex comprises a lipid bilayer shaped in a spherical vesicle form, in which the lipid bilayer envelops a drug within the internal space of the vesicle. For example, the particular chemistry and geometry of liposomes enable an efficient delivery system that can simultaneously reduce the toxicity of therapeutics as well as enhancing the potency of the drug. The therapeutic index of the drug can be modulated in order to reduce the toxicity and/or increase the efficacy of the parent drug (Bangham A D, Liposomes: the Babraham connection, Chem. Phys. Lipids 64:275-285 [1993]). Similar liposome-based therapeutics have been approved for human use by the U. S. Food and Drug Administration (“FDA”). Thus, liposomes have been used as drug carriers in pharmaceutical applications since the mid-1990s (Lian, T. and Ho, R. J. Y., Trends and Developments in Liposome Drug Delivery Systems, J. Pharm. Sci. 90(6):667-80 [2001]).

Liposomes can be designed to have more stability both in vitro and in vivo, with improved biodistribution, and with optimized resident time of liposomes in the systemic circulation. By utilizing hydrophilic polymers to enhance the degree of surface hydration or by using steric modification strategies, the surface of a liposome membrane can be modified in order to reduce the degree of aggregation and to avoid recognition by RES cells. For example, surface modification is often done by incorporating gangliosides, such as GM₁, or lipids that are chemically conjugated to hygroscopic or hydrophilic polymers, usually polyethyleneglycol (“PEG”). Similar to the process of protein PEGylation, in which PEG molecules are conjugated to therapeutic proteins such as adenosine deaminase, a common alderase used for the treatment of AIDS, PEG can be conjugated to the terminal amine of phosphatidylethanolamine constituting a liposome (Beauchamp C, et al., Properties of a novel PEG derivative of calf adenosine deaminase, Adv Exp Med Biol 165:47-52 [1984]). The presence of hydrophilic polymers on the liposome membrane surface provides an additional surface hydration layer (Torchilin V P, Immunoliposomes and PEGylated immunoliposomes: possible use of targeted delivery of imaging agents, Immunomethods 4:244-258 [1994]). One advantage of hydrated liposomes is that such liposomes evade recognition by macrophages and RES cells as foreign particles, and therefore, precludes phagocytic clearance by these cells.

Liposome size may affect vesicle distribution and clearance after systemic administration so that increasing the vesicle size can enhance RES-mediated uptake of liposomes (Hwang K, Liposome pharmacokinetics, In: Ostro M J, editor, Liposomes: from biophysics to therapeutics, New York: Marcel Dekker, pp. 109-156 [1987]). Whereas RES-mediated uptake in vivo can be saturated at high doses of liposomes or by pre-dosing with large quantities of control liposomes deficient in drug content, this strategy may not be practical for human therapeutic use because sustained impairment of the RES physiological functions may introduce adverse effects in patients (Senior J, et al., Tissue distribution of liposomes exhibiting long half-lives in the circulation after intravenous injection, Biochem Biophys Acta 839:1-8 [1985]). Most recent investigations have employed unilamellar vesicles, 50-100 nm in size, for systemic drug delivery applications. For example, the antifungal liposome product “AmBisome” can be formulated so that the size specification is 45-80 nm in order to reduce the RES-mediated uptake of antifungal liposomes.

Serum protein binding is an important factor that affects liposome size and increases the rate of liposome clearance in vivo, when administered by intravenous (IV) route. In particular, processes such as complement activation by liposomes and opsonization depend on liposome size (Devine D V, et al., Liposome-complement interactions in rat serum: Implications for liposome survival studies, Biochim Biophys Acta 1191:43-51 [1994]; Liu D, et al., Recognition and clearance of liposomes containing phosphatidylserine are mediated by serum opsonin, Biochem Biophys Acta 1235:140-146 [1995]). Although PEG can be incorporated into liposome formulations to reduce serum protein binding to liposomes, the upper size limit to ensure prolonged circulation of PEG-PE liposomes is ˜200 nm. Due to biological constraints, the development of relatively large (>500 nm) liposomal structures having prolonged circulating properties, by using steric stabilization methods, has not been successful. For optimizing liposome-drug delivery systems, liposome composition and size are critical considerations in that the mechanisms of biodistribution and disposition in vivo can vary depending on the lipid composition, the liposome size, the liposome charge, and the degree of liposome surface hydration or steric hindrance.

The route of administration may affect the in vivo disposition of liposomes mainly because immediately after intravenous administration, liposomes are usually coated with serum proteins, and are taken up or eliminated by circulating RES cells (Chonn A, et al., Association of blood proteins with large unilamellar liposomes in vivo. Relation to circulation lifetimes, J Biol Chem 267:18759-18765 [1992]; Rao M, et al., Delivery of lipids and liposomal proteins to the cytoplasm and Golgi of antigen-presenting cells, Adv Drug Deliv Rev 41:171-188 [2000]). Plasma proteins that can interact with liposomes include albumin, lipoproteins or any high-density lipoprotein (“HDL”), low-density lipoprotein (“LDL”) and cell-associated proteins. Some of these proteins such as HDL can remove phospholipids from the liposome bilayer, thereby destabilizing the liposomes. This process may potentially lead to a premature leakage or dissociation of drugs from liposomes.

As a drug delivery system, liposomes are especially promising because they can modulate the pharmacokinetics of liposome-associated and encapsulated drugs, which is not possible with non-lipid-associated or “free” drugs (Allen, T. M et al. [1991]; Hwang, K. [1987]; Allen T, et al, Pharmacokinetics of long-circulating liposomes, Adv Drug Del Rev 16:267-284 [1995]; Gabizon A, Liposome circulation time and tumor targeting: Implications for cancer chemotherapy, Adv Drug Del Rev 16:285-294 [1995]; Bethune C, et al., Lipid association increases the potency against primary medulloblastoma cells and systemic exposure of 1-(2-chloroethyl)-3-cyclohexyl-1-nitrosourea (CCNU) in rats, Pharm Res 16:896-903 [1999]). However, therapeutic applications of systemically (IV) administered liposomes have been limited by the rapid clearance of liposomes from the bloodstream and uptake by RES cells (Alving C, et al., Complement-dependent phagocytosis of liposomes: Suppression by ‘stealth’ lipids, J Liposome Res 2:383-395 [1992]). Incorporation efficiencies for loading many pH-titratable drugs within the interior aqueous compartment of liposomes, including some protease inhibitors such as indinavir, typically have been relatively low (Gagne, J F et al., Targeted delivery of indinavir to HIV-1 primary reservoirs with immunoliposomes, Biochim Biophys Acta 1558(2):198-210 [2002]). Although significant advances have been made in the field of lipid-drug formulation technology in recent years, a need for developing compositions and methods that can provide an effective pharmaceutical-delivery system, which can incorporate drugs and biomolecules or “biologicals” at high efficiency, and deliver stable lipid-pharmaceutical and lipid-biological complexes to a lymphoid tissue is recognized.

### SUMMARY

The present application is directed to lipid-drug complexes and related methods for producing stable lipid-drug complexes at or near the neutral pH range and administering pharmaceutical lipid-drug complexes to patients. In certain examples, the lipid-drug complex has a lipid-to-drug molar ratio of approximately 3:1. In certain examples, the lipid-drug complex may have lipid-to-drug molar ratios of less than 3:1 to 10:1 or higher. The present application is also directed to methods of administering a drug to a patient though subcutaneous injection of the lipid-drug complexes into particular tissues to effect higher localized concentrations of the at least one drug. Lipid-anti-HIV-drug complexes can be subcutaneously injected into the lymphoid tissue of a HIV-infected mammalian subject via the lymphatic vessels to deliver high concentrations of stable lipid-anti-HIV-drug complexes, rather than delivery of the anti-HIV drug intravenously via the blood stream which will eventually reach the lymphatic system at lower concentrations.

### BRIEF DESCRIPTION OF THE DRAWINGS

FIG. 1A illustrates the pH-dependent incorporation of indinavir within lipid-associated complexes, as discussed in Example 2.

FIG. 1B illustrates the pH-dependent release of indinavir from lipid-associated complexes in vivo, as discussed in Example 2.

FIG. 2A illustrates a typical time course for a virus load, and the CD4⁺ T cell profile of macaques infected with HIV-2₂₈₇, as discussed in Example 3.

FIG. 2B illustrates the analysis of plasma for a viral RNA profile of 27 macaques that were infected with 50 TC_{ID5O} HIV-2₂₈₇, as discussed in Example 4.

FIG. 3 illustrates the concentration-dependent inhibition of HIV-₂₈₇ replication by free (not lipid-associated) and lipid-associated indinavir, as described in Example 5.

FIG. 4 illustrates a time course for plasma concentration of indinavir following the subcutaneous administration of lipid-associated and non-lipid-associated indinavir within macaques, as described in Examples 6 and 7.

FIGS. 5A-B illustrates the changes in plasma virus load and the CD4⁺ T-cell profile monitored in two HIV-2₂₈₇-infected macaques at 25 weeks post-infection, as described in Example 8.

FIG. 6 shows the concentration-dependent inhibition of HIV-1_{LAV} replication by the free and lipid-associated indinavir, as described in Examples 5 and 9.

FIGS. 7A-B shows in situ hybridization analysis of lymph node sections in indinavir treated animals with a [³⁵S]-labeled HIV-2₂₈₇-specific probe, as described in Example 10.

FIG. 8 illustrates the drug-incorporation and drug-release characteristics of lipid-drug complexes to which the present application is directed.

FIG. 9 provides a table showing characteristics of the drugs indinavir and tenofovir and the dye calcein.

FIG. 10 provides a plot of fluorescence polarization versus temperature, as measured by using a diphenylhexatriene (“DPH”) fluorescence membrane probe, for indinavir incorporated within the hydrophobic domain of a phospholipid bilayer.

### DETAILED DESCRIPTION

The present application is directed to various pH-sensitive lipid-drug complexes, methods for producing the lipid-drug complexes, and methods for employing lipid-drug complexes in order to treat various clinical conditions, including diseases that affect lymphoid tissues. The formation of lipid-drug complexes may be referred to as “lipid association” or “lipid incorporation.” The reverse process of dissociating a lipid-drug complex may be described as “dissociation” or “drug release.”

Some examples of the lipid-drug complexes to which the present application is directed include lipid-drug-complex formulations and methods for efficiently incorporating anti-HIV drugs into lipid-drug delivery vehicles. Lipid-anti-HIV-drug complexes can be subcutaneously injected into the lymphoid tissue of a HIV-infected mammalian subject via the lymphatic vessels to deliver high concentrations of stable lipid-anti-HIV-drug complexes, rather than delivery of the anti-HIV drug intravenously via the blood stream which will eventually reach the lymphatic system at lower concentrations.

HIV-infected cells exposed to anti-HIV drugs delivered by lipid-drug complexes may uptake a greater amount of the anti-HIV drugs than HIV-infected cells exposed to non-lipid-associated, free anti-HIV drug. Therefore, smaller amounts of lipid-associated drugs can be administered than the amounts of free anti-HIV drugs needed to provide therapeutic benefit by systemic administration. The lipid-anti-HIV-drug complexes disclosed in the present application provide for targeting HIV reservoirs within infected lymphoid cells located within the lymph nodes. The lipid-anti-HIV-drug complexes disclosed in the present application can be administered less frequently than free anti-HIV drugs while providing comparable therapeutic benefits. Lipid-drug complexes similar to those disclosed in the present application can also be used to administer other types of drugs to treat other types of diseases.

The lipid-drug complexes to which the present application is directed may adopt various types of configurations, including spherical configurations of liposomes, various lipid-drug-sheet-disk complexes, and other configurations. A liposome forms generally as a vesicle comprising a lipid bilayer membrane with an aqueous internal space. Other types of lipid-drug complexes have non-vesicular bilayer configurations or micelle-like configurations. For many applications, liposome-drug complex are desired to have diameters ranging from 30 to about 150 nanometers or ranging from 50 to about 80 nanometers (see Table 1B, Example 2).

The drug component of various lipid-drug complexes to which the present application is directed is an anti-viral drug, such as a non-nucleoside anti-HIV drug. Examples of non-nucleoside anti-HIV drugs include the HIV reverse protease inhibitors: indinavir (Crixivan®, Merck & Co., Inc., Rahway, N.J.); saquinavir (Fortovase®, Roche Laboratories, Inc., Nutley, N.J.), N-tert-butyl-decahydro-2-[2(R)-hydroxy-4-phenyl-3(S)-[[N-(2-quinolylcarbonyl)-L-asparaginyl]-amino]butyl]-(4aS,8aS)-isoquinoline-3 (S)-carboxamide, MW=670.86; or nelfinavir (nelfinavir mesylate) (Viracept®Pfizer formally, Agouron Pharmaceuticals, Inc., La Jolla, Calif.), [3S-[2(2S*, 3S*), 3a,4ab,8ab]]-N-(1,1-dimethylethyl)decahydro-2-[2-hydroxy-3-[(3-hydroxy-2-methylbenzoyl)amino]-4-(phenylthio)butyl]-3-isoquinolinecarboxamide mono-methanesulfonate (salt), MW=663.90 (567.79 as the free base). Other examples of antiviral drugs include reverse transcriptase inhibitors, such as tenofovir disoproxil fumarate (Viread®, Gilead Sciences, Foster City, Calif.), 9-[(R)-2-[[bis[[(isopropoxycarbonyl)oxy]methoxy]phosphinyl]methoxy]propyl]adenine fumarate (1:1), MW=635.52.

The drug component of additional various lipid-drug complexes, to which the present application is directed, is an anticancer drug, an antifungal drug, an antibacterial drug, or an immunomodulatory drug (i.e., an immunoactivator, an immunosuppressant, or an antiinflammatory), such as cyclosporin, steroids and steroid derivatives. Lipid-drug complexes to which the present application is directed are produced by lipid incorporation or lipid association of a number different types of drugs and combinations of drugs. For example, liposomes can incorporate a large number of one or more different anti-HIV drugs, anti-fungal drugs, antibacterial drugs, and anti-cancer drugs.

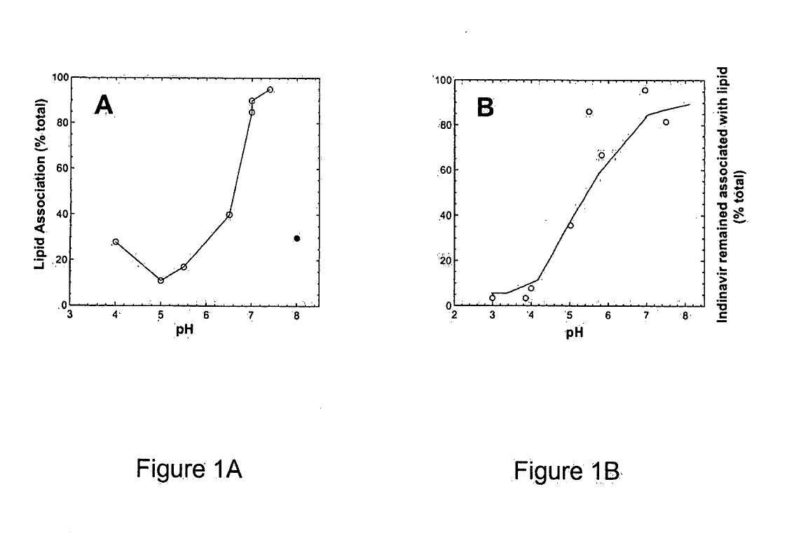

The lipid-drug complexes to which the present application is directed provide a lipid-based drug-delivery vehicle for drugs that exhibit an increase in membrane affinity within a neutral or physiological pH range. These drugs are generally characterized by low aqueous solubility within a neutral pH range of pH 5.5 to about pH 8.0 or, alternatively, from pH 7.0 to about pH 7.4 (see FIGS. 1A and 1B). Drugs such as indinavir, nelfinavir, saquinavir, viread (described above and see Table 1B, Example 2) are examples of drugs with low aqueous solubility and increased lipohilicity within a neutral pH range. Nelfinavir mesylate, as one example, is a white to off-white amorphous powder which is slightly soluble in water at pH<4 and is freely soluble in methanol, ethanol, isopropanol and propylene glycol.

In many cases, lipohilic drugs with low solubility at neutral pH, such as indinavir, intercalate within lipid bilayers that form when amphiphilic molecules, including phosphatidylcholines and carboxylic acids with long aliphatic tails, are introduced into aqueous media. The drug indinavir includes an aniline moiety with a relatively low dissociation constant (pK=4.76) indicative of a weak acid. Protonation of the aniline amine at low pH introduces a positive charge within the drug that, in turn, results in increased aqueous solubility of the drug. Titration back to a neutral pH range decreases the aqueous solubility of the drug and increases its lipophilicity, resulting in intercalation of the drug within a lipid bilayer present in the solution, including the lipid bilayer of a liposome present in the solution. It is thought that endocytosis or internalization of liposomes by cells results in sequestration of indinavir within intracellular acidic vesicles and that the acidic pH within these vesicles increases the aqueous solubility of the drug, resulting in its release from the liposome into the cell.

By way of example, indinavir is an HIV protease inhibitor, typically formulated as a sulfate salt of N-(2(R)-hydroxy-1(S)-indanyl)-2(R)-phenylmethyl-4-(S)-hydroxy-5-(1-(4-(3-pyridyl-methyl)-2(S)-N′-(t-butylcarboxamido)-piperazinyl))-pentaneamide ethanolate. Indinavir in pill form is typically administered to AIDS patients at a dosage of 800 mg, three times a day. Indinavir is commonly taken in pill form and therefore delivered systemically to lymphoid tissues. Indinavir has about 1000-fold lower solubility in water at neutral pH 7 than at acidic pH 3-4. However, by formulating a lipid-indinavir complex, at a lipid-to-drug molar ratio range from about 5:1 to about 10:1, within a neutral pH range where the aqueous solubility of indinavir is relatively low, 80-100% of an indinavir preparation is incorporated, whereas, at pH 3-4, much lower incorporation efficiencies of less than 30% are obtained.

Certain of the currently disclosed methods for preparing indinavir involve is dissolving the drug in a solvent. In some cases, the drug can be dissolved in an aqueous solvent, such as water or a biocompatible buffer solution, including phosphate-buffered saline, HEPES, TRIS, or the like. A detergent, such as Tween 80, can also be employed in conjunction with an aqueous solvent. Dissolving the drug can be accomplished in the presence, in the absence, or before the addition of the lipids. In other cases, the drug is more effectively dissolved in an organic solvent, such as dimethyl sulfoxide (DMSO), methanol, ethanol, propanol, propane glycol, butanol, isopropanol, pentanol, pentane, a fluorocarbon, or an ether.

Examples of useful lipids include any vesicle-forming lipid, such as, but not limited to, phospholipids, such as phosphatidylcholine (“PC”), both naturally occurring and synthetically prepared, phosphatidic acid (“PA”), lysophosphatidylcholine, phosphatidylserine (“PS”), phosphatidylethanolamine (“PE”), and sphingolipids, phosphatidyglycerol (“PG”), spingomyelin, cardiolipin, glycolipids, gangliosides, cerebrosides and the like used either singularly or intermixed such as in soybean phospholipids (e.g., Asolectin, Associated Concentrates). The PC, PG, PA and PE can be derived from purified egg yolk and its hydrogenated derivatives.

In addition, other lipids such as steroids, cholesterol, aliphatic amines such as long-chained aliphatic amines and carboxylic acids, long-chained sulfates and phosphates, diacetyl phosphate, butylated hydroxytoluene, tocopherols, retinols, and isoprenoid compounds can be intermixed with the phospholipid components to confer certain desired and known properties onto the formed vesicles. In addition, synthetic phospholipids containing either altered aliphatic portions such as hydroxyl groups, branched carbon chains, cycloderivatives, aromatic derivatives, ethers, amides, polyunsaturated derivatives, halogenated derivatives or altered hydrophilic portions containing carbohydrate, glycol, phosphate, phosphonate, quarternary amine, sulfate, sulfonate, carboxy, amine, sulfhydryl, or imidazole groups. Combinations of such groups can be either substituted or intermixed with the above-mentioned phospholipids. It will be appreciated from the above that the chemical composition of the lipid components prepared by the present method can be varied greatly without appreciable diminution of percentage drug capture, although the size of a vesicle can be affected by the lipid composition. Saturated synthetic PC and PG, such as dipalmitoyl can also be used. Other amphipathic lipids that can be used, advantageously with PC, include gangliosides, globosides, fatty acids, stearylamine, long chain alcohols, and the like. PEGylated lipids, monoglycerides, diglycerides, triglycerides can also be included. Acylated and diacylated phospholipids are also useful. Useful phospholipids also include egg phosphatidylcholine (“EPC”), dilauryloylphosphatidylcholine (“DLPC”), dimyristoylphosphatidylcholine (“DOPC”), dipalmitoylphosphatidylcholine (“DPPC”), distearoylphosphatidylcholine (“DSPC”), 1-myristoyl-2-palmitoylphosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dioleoylphosphatidylycholine (“DOPC”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), distearoyl sphingomyelin (“DSSP”), distearoylphophatidylethanolamine (DSPE), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), and dipalmitoyl sphingomyelin (“DPSP”). In one example lipid-drug complex, phosphatidylcholine and cholesterol at a molar ratio of 3:1 are employed. However, any suitable molar ratio of a non-steroidal, lipid-steroidal lipid (e.g., cholesterol) mixture can optionally be employed that promotes the stability of a particular lipid-drug complex during storage and/or delivery to a mammalian subject.

Mixing the drug and lipids can be by any useful known technique, for example, by sonication, vortexing, extrusion, microfluidization, homogenization, and use of a detergent, which may be later removed, e.g., by dialysis. The drug and lipid are mixed at a lipid-to-drug molar ratio of about 3:1 to about 100:1 or higher, where larger molar ratios are useful for relatively more toxic drugs, at a lipid-to-drug molar ratio of about 3:1 to about 10:1, or at a lipid-to-drug molar ratio of about 5:1 to about 7:1. When an organic solvent is used for producing a lipid-drug complex, such as a liposome, the organic solvent can be removed, after the mixing of the drug and lipids, by any suitable means of removal, such as evaporating by vacuum, by the application of heat by, for example, using a hair dryer or an oven, or by hot ethanol injection, as long as the lipid and drug components are stable at the temperature used. Dialysis and/or chromatography, including affinity chromatography can also be employed to remove the organic solvent. Drug hydration is performed with water or any biocompatible aqueous buffer, e.g., phosphate-buffered saline, HEPES, or TRIS, that maintains a physiologically balanced osmolarity. Rehydration of liposomes can be accomplished by simultaneously removing the organic solvent, or alternatively, can be delayed until a more convenient time for using the liposomes. The shelf life of hydratable (i.e., “dry”) liposomes is typically about 8 months to about a year, which can be increased by lyophilization.

The lipid-drug complexes to which the present application is directed include a lipid bilayer or other stable lipid phase in which a lipophilic drug is incorporated at neutral pH. The molar ratio of the lipid to drug in the lipid-drug complex is within a range of about 3:1 to about 100:1 or higher, within a range of about 3:1 to about 10:1, or within a range of 5:1 to about 7:1. The lipid-drug complexes are also characterized by the fact that the drug substantially dissociates from the lipid-drug complexes at a range of pH from about pH 5.0 to about pH 5.5. The phrase “substantially dissociates” means that approximately 50% or more of the drug that is associated with the lipid in the lipid-drug complex at neutral pH dissociates from the lipid-drug complex when the pH is lowered to a pH at or below 5.5.

The lipid-drug complex is administered to a subject by any suitable means, for example, by injection. Injection can be intrarterial, intravenous, intrathecal, intraocular, subcutaneous, intramuscular, intraperitoneal, or by direct (e.g., stereotactic) injection into a tumor or other types of lesion. Subcutaneous or intramuscular injection can be used for introducing the lipid-drug complex into lymphatic vessels. The lymphoid tissue is a lymph node, such as an inguinal, mesenteric, ileocecal, or axillary lymph node, or the spleen, thymus, or mucosal-associated lymphoid tissue (e.g., in the lung, lamina propria of the of the intestinal wall, Peyer's patches of the small intestine, or lingual, palatine and pharyngeal tonsils, or Waldeyer's neck ring). Injection can be performed by any method that drains directly into the lymphatic system as opposed to into the blood stream. Commonly, administration is by way of subcutaneous injection, typically employing a syringe needle with a gauge larger than the diameter of the lipid-drug complex. Intraperitoneal injection can also be used. Typically, the injectate volume (generally about 1-5 cm³) is injected into the subject's arm, leg, or belly, but any convenient site can be chosen for subcutaneous injection. Because the drug is subcutaneously administered, the drug enters the lymphatic system prior to entering the systemic blood circulation, providing: (1) distribution throughout the lymphoid system and localization into lymph nodes due to particle filtration and fenestration, (2) preclusion or minimization of protein-mediated destabilization of lipid-drug complexes, and (3) delivery of the drug at concentrations that cannot be achieved with a soluble form of the drug administered by any route of administration.

Typically, for methods directed to treating HIV/AIDS, the frequency of injection is most preferably once per week, but more or less (e.g., monthly) frequent injections can be given as appropriate. The lipid-drug complexes to which the present application is directed facilitate a treatment regimen that can involve a convenient weekly injection rather than multiple drug doses daily, as practiced typically in current AIDS treatment regimes. This feature may lead to improved patient compliance with the full course of treatment for some individual patients. See Snedecor S J, Sullivan S M, Ho R J Feasibility of weekly HIV drug delivery to enhance drug localization in lymphoid tissues based on pharmacokinetic models of lipid-associated indinavir. Pharm Res. 2006 August;23(8):1750-5.

The lipid-drug complexes to which the present application is directed are further described by following examples. In Example 1, methods employed to prepare certain of the lipid-drug complexes to which the present application is directed are provided. In Example 2, experimental data supporting the pH-dependence of lipid-drug association/incorporation efficiency is provided along with data supporting the pH-dependent efficiency of drug release from lipid-associated complexes. Also, in Example 2, the effect of pH on the solubility and lipophilicity of an example drug (indinavir) is provided (Table 1A as well as the relative sizes and degrees of lipid association for various types of drugs (Table 1B). In Example 3, data supporting enhanced levels of drug delivery to lymphoid tissues are provided by comparing indinavir concentrations in human lymph node (LNMC) and peripheral blood mononuclear cells (PBMCs). In Example 3, a typical time course following HIV-2 infection in monkeys is presented (FIG. 1A). In Example 4, a time course for plasma concentration of indinavir following the subcutaneous administration of lipid-associated and non-lipid-associated indinavir within macaques is provided. In Example 5, the effect of lipid association on the ability of indinavir to inhibit HIV-2₂₅₇ replication is provided. In Example 6, a plasma time course profile of free versus lipid-associated indinavir in macaques is provided. In Example 7, the effect of lipid-drug complexes on enhanced accumulation of indinavir in lymph nodes is provided. In Example 8, the effect of lipid-indinavir complex on HIV-2₂₈₇ infected macaques is provided. In Example 9, the effect of lipid association on the inhibition of HIV-I replication in human peripheral blood mononuclear cells is provided. In Example 10, the reduction of HIV viral load in infected macaques by the accumulation of liposome-indinavir complexes in lymphoid tissues is provided.

### EXAMPLES

### Example 1

Lipid-drug complex preparation and characterization: Routinely, for drug incorporation studies, 1 millimole of the drug (e.g., indinavir, saquinavir, nelfinavir, or tenofovir disoproxil fumarate) was dissolved in 1 mL of ethanol and mixed together with 5 mmoles of lipids (e.g., phosphatidylcholine [egg]: cholesterol [3:1, mol/mol]) dissolved in CHCl₃:ethanol (1:1, v/v). The mixture was rotor-evaporated under N₂ and reduced pressure. Then it was resuspended in sterile phosphate-buffered saline (PBS, pH 7.4), to a final lipid concentration of 50 mM, and homogenized with a probe sonicator until a uniform particle size of about 50 nm to about 80 nm diameter was reached. Sonication is preferably done until sonicated unilamellar vesicles that are stable at their minimum diameter are obtained. Typically, it took less than 30 min of sonication under aseptic conditions to achieve a 50-60 nm lipid-drug complex size in a relatively narrow distribution. The size and zeta potential (surface potential at hydrodynamic plane) of the lipid-drug complex was monitored using a Malvern Zetasizer 5000 operating at photon correlation spectroscopy mode and electrophoretic mode, respectively. In the case where a negative membrane charge is needed, about 20 mole % of phosphatidylglycerol (exhibits a net negative charge at neutral pH) can be added to the lipid mixture, solubilized in CHCl₃:ethanol (1:1, v/v). The rest of the preparation procedure remains the same.

The sterility and endotoxin contamination of the preparation, an important safety consideration, was routinely monitored as part of strict cGMP/cGLP guidelines. To ensure sterility and prevent underestimation of endotoxin that binds to lipids, the lipid-drug complex preparations were subjected to the blood agar culture test for 7 days at 37° C. for detection of microbial contamination. This provided a stringent evaluation of sterility and bacterial contamination to ensure the consistent quality of the lipid-drug complex.

The degree of drug incorporation into the lipid-drug complex was determined by subjecting a small fraction of the preparation to size-exclusion chromatography using a Biogel A-0.5 M media (1×10 cm). At the flow rate of 1 ml/min of PBS, lipid-associated indinavir was well separated from free drug. By analyzing the amount of fraction in the lipid-associated complex, and free form with respect to the total amount loaded onto the column, the percentage of drug association was determined. (Lian, T. and Ho, R. J. Y., Recent trend and progress in liposome drug delivery systems; an invited minireview, J Pharm Sci 90:667-680 [2001]). To determine pH-dependent drug dissociation from the lipid-drug complex, the lipid-drug complex (originally prepared in pH 7.4 and free of unincorporated drug) can be incubated at, e.g., pH 7, 6, 5.5, 5, 4, 3.5 for 30 min. Subsequently, the mixture can be subjected to the degree of drug incorporation analysis described above. For animal studies, the drug-lipid complex formulations were further analyzed for sterility and endotoxin contamination. To ensure sterility and prevent underestimation of endotoxin that binds to lipids, the lipid-drug complex preparations were subjected to a blood agar culture test for 7 days at 37° C. for detection of microbial contamination.

Determination of virus-infected cell frequency in peripheral blood and lymph node mononuclear cells by virus coculture assay: From the macaques, PBMCs were isolated from 10-ml blood samples at twice-weekly intervals after HIV-2₂₈₇ inoculation. About 1-2×10⁶ PBMCs/ml of blood were routinely isolated, allowing the performance of coculture assays that minimally require about 1×10⁶ PBMCs.

HIV-2₂₈₇ was originally isolated from the lymph node of a macaque with the clinical manifestations of AIDS. The macaque had been inoculated with HIV-2_{EHO} which had been passaged twice in macaques. A stock was prepared by growing the primary isolate in CD8⁺-depleted, phytohemagglutinin-stimulated macaque PBMC. (Ho R J, Agy M B, Morton W R, et al. Development of a chronically catheterized maternal-fetal macaque model to study in utero mother-to-fetus HIV transmission: a preliminary report. J Med Primatol 1996;25 (3):218-24; Ho R J, Larsen K, Kinman L, et al. Characterization of a maternal-fetal HIV transmission model using pregnant macaques infected with HIV-2(287). J Med Primatol 2001;30 (3):131-40). The inoculum was prepared by diluting the stock in complete tissue culture medium.

Briefly, duplicate samples of the serially diluted PBMCs isolated from the blood in a fixed volume (2 ml) were added to 10⁶ human lymphoblasts in 24-well tissue culture plates. The human lymphoblasts were generated by first depleting CD8⁺ cells from the PBMCs and then stimulating the lymphocytes (i.e., PBMCs) for 3 days with 1 μg/ml PHA-P and 20 IU/ml IL-2 in RPMI containing 10% NHu serum. The CD8⁺ cell-depleted macaque PBMCs were sequentially diluted (in 1:5 ratios) starting from 10⁶ cells per well. The feeder cells, human lymphoblasts, remained constant at 10⁶ per well. The cell mixture was incubated for 21 days with the cells being fed with fresh culture medium once every week. The presence of HIV-infected macaque PBMCs was visually screened every other day for syncytia formation and verified for infection on days 14 and 21 by assaying for the HIV-2 p27 core antigen in supernatant using a sandwich antigen ELISA. The end-point dilution at which minimally detectable HIV antigen is found can be used to estimate the frequency of virus-infected cells per 10⁶ macaque PBMCs. Typically, by about day 10-13 following infection with a dose of 10 TC_{ID}_{₅₀} HIV-2₂₈₇, the virus-infected cells peaked at about 25,000 per 10⁶ PBMCs, an extremely high frequency of virus-infected cells (˜0.1-1% of total PBMCs) in the periphery. (Ho, R. J. Y. et al., Suppression of maternal virus load with AZT, DDI, and indinavir combination therapy prevents mother-to-fetus HIV transmission in macaques, JAIDS, 25, 140-149 [2000]; Ho, R. J. Y. et al., Development of a chronically catheterized maternal-fetal macaque model to study in utero mother-to-fetus HIV transmission—a preliminary report. An invited article, Medical Primatol 25, 218-224 [1996]).

Analysis of viral RNA and DNA in blood, lymph nodes, and other tissues: Tissues collected (lymph nodes, thymus and spleen, brain) from macaques at the time of euthanasia were assayed for drug level, as described herein below, and analyzed by immunohistochemistry, RNA- and DNA-PCR to quantitate viral load and distribution of virus in these tissues. For DNA- and RNA-PCR analyses as well as virus co-culture, fresh or flash-frozen (stored at −80° C.) tissues were used. For immunocytochemistry, in situ hybridization, and other histological analyses were conducted; the tissues were fixed following established procedures.

Briefly, tissues were be fixed in 4% neutral buffered and deionized paraformaldehyde, were embedded in paraffin wax, sectioned (5 μm) and stained with hematoxylin and eosin for routine histological examination. In addition, lymph node tissues were preserved in Streck Tissue Fixative (STF; Streck Laboratories, Omaha, Nebr.), a citrate-based, non-cross-linking fixative suitable for permeating dense tissues, maintaining the integrity of nucleic acids, and conserving antigenic structure of cell-surface molecules. A fraction of the tissues were used to isolate lymph node leukocytes by forcing the tissues through an 80-μm wire mesh and layering onto histopaque 1077 (Sigma, St. Louis, Mo.) discontinuous density gradients to isolate PBMC or lymph node mononuclear cells (LNMC) (Brodie, S. J., et al., Pediatric AIDS-associated lymphocytic interstitial pneumonia and pulmonary arterio-occlusive disease: Role of VCAM-1/VLA-4 adhesion pathway and human herpesviruses, Am J Pathol 154:1453-1464 [1999a]; Brodie, S. J. et al., In vivo migration and function of transferred HIV-1-specific cytotoxic T cells, Nat Med 5(1):34-41 [1999b]; Brodie, S. J. et al, HIV-specific cytotoxic T lymphocytes traffic to lymph nodes and localize at sites of HIV replication and cell death, J Clin Invest 105:1407-1417 [2000b]). LMNC and PBMC were fixed and permeated (to preserve intracellular nucleic acid) with Permeafix (Ortho Diagnostics, Raritan, N.J.) (500 μl/10⁶ cells), a non-aldehyde, non-cross-linking, water-soluble fixative.

Total RNA was isolated from plasma and lymphoid tissues using a Purescript RNA Isolation Kit (Gentra Systems, Minneapolis, Minn.). Viral RNA was measured using a quantitative, internally-controlled RNA PCR to estimate the number of HIV-2 copies/ml of sample (Watson A, Ranchalis J, Travis B, et al., Plasma viremia in macaques infected with simian immunodeficiency virus: plasma viral load early in infection predicts survival, J Virol 1997;71 (1):284-90) for some of the experiments and real-time quantitative PCR. The two methods have been validated to ensure consistency in estimating viral RNA concentration in plasma and tissues. Briefly, reverse transcription and PCR were performed as described. Because semi-quantitative methods have been well-documented (Watson, et al., 1997), only details on RT-QPCR are described below.

Real-time quantitative fluorescent probe PCR (TaqMan): The TaqMan PCR system was employed in a real-time automated PCR assay for quantifying HIV-1 or HIV-2 (e.g., HIV-2₂₈₇) DNA and RNA in plasma, PBMC, and/or a variety of solid tissues for viral load determination in HIV-infected subjects. (Brodie et al., [2000a-b]; Mostad, S. B. et al., Cervical shedding of cytomegalovirus in human immunodeficiency virus type 1-infected women, J Med Virol 59:469-473 [1999]; Mostad, S. B. et al., Cervical shedding of herpes simplex virus in human immunodeficiency virus-infected women: effects of hormonal contraception, pregnancy, and vitamin A deficiency, J Infect Dis 181:58-63 [2000]; Krone, M. R. et al, Herpes simplex virus type 2 shedding in human immunodeficiency virus-negative men who have sex with men: frequency, patterns, and risk factors, Clin Infect Dis 30:261-267 [2000]; Wald, A. et al., Reactivation of genital herpes simplex virus type 2 infection in asymptomatic seropositive persons, N Engl J Med 342:844-50 [2000]; Zerr, D. M. et al., Sensitive method for detection and quantification of human herpesviruses 6 and 7 in saliva collected in field studies, J Clin Microbiol. 38(5):1981-83 [2000]; Ryncarz, A. J. et al, Development of a high-throughput quantitative assay for detecting herpes simplex virus DNA in clinical samples, J Clin Microbiol 37:1941-1947 [1999]). The real-time RT-PCR assay for detecting HIV gag RNA has been validated.

Real-time PCR is a technology that offers advantages over conventional methods, including quantitation of product copy numbers following each PCR cycle (facilitating mathematical calculation of precise copy numbers in the original sample) and markedly reduced susceptibility to contamination. This technology is highly quantitative and reproducible, and allows the consistency to perform a large volume of sample collections.

DNA and RNA were extracted from 400 μl of plasma using acid phenol (pH 4): chloroform: isoamyl alcohol (48:24:1). The specimens were eluted into 100 μl of 10 mM Tris (pH 8.0) and 20 μl of nucleic acid was used for each PCR and RT-PCR reaction. Tissues and cells were first treated with proteinase K before extraction. One to 2 μg of total cellular DNA or RNA were used for each PCR. For RNA, the nucleic acid was reverse-transcribed and amplified in a one-step reaction (Perkin Elmer, Multiscribe) (Brodie et al., 2000b). The conditions and controls for the TaqMan PCR were similar to those described in Brodie et al. (2000a-b). The design of specific primers and fluorescent probes for HIV-2 gag DNA and RNA were based on HIV-2_{EHO} sequences (Rey-Cuille, M. A. et al., HIV-2_{EHO} isolate has a divergent envelope gene and induces single cell killing by apoptosis, Virology 202:471-6 [1994]):

Primers
(SEQ ID NO: 1)
EHOTAQ-F′: TTATTCCCACCTGCCGCTAA;
(SEQ ID NO: 2)
EHOTAQ-R′: CTGCCCCGAACTTCTTCTCTT;
and
Probe
(SEQ ID NO: 3)
EHOTAQ-P′: CCCCCAACCTTAAATGCCTGGG.

Liquid hybridization PCR: A semiquantitative liquid hybridization PCR assay was also used to detect HIV-2, as a ‘confirmation’ assay to real-time PCR methods. The assay is capable of detecting a single virus copy per sample and is similar to what has been reported for HIV-1. (Brodie et al. [1999] and [2000b]). Nucleic acid was extracted from proteinase K-treated tissues. For vRNA, the nucleic acid was heated, cooled, and cDNA was synthesized using random hexamer primers. Sequence-specific primers were used to amplify the cDNA and the amplified viral sequence was subsequently detected by liquid hybridization using a [³²P]-labeled oligonucleotide probe specific for a conserved internal region of the amplified viral product. Electrophoresis was performed in a 6% polyacrylamide gel, and the gel was dried for autoradiography. Each autoradiograph band was compared with a dilution curve containing 5, 50, 500, and 5000 copies of viral RNA, respectively. Each cDNA and PCR reaction contained both positive and negative controls. All samples that were PCR-negative for virus were confirmed to be inhibitory or non-inhibitory by performing an additional PCR with 10³ copies of viral cDNA. Samples that failed to support amplification of the input substrate were denoted as inhibitory. All others were reported as samples void of viral RNA. Viral copy numbers were determined using the computer program QUALITY, which is based on the number of amplifications and the number of positive results at each dilution (Rodrigo, A. G. et al., Quantitation of target molecules from polymerase chain reaction-based limiting dilution assays, AIDS Res Hum Retroviruses 13:737-42 [1997]).

Localization of HIV-2 DNA and RNA in tissue sections using PCR in-situ hybridization (PCR-ISH) and in-situ hybridization (ISH): Biopsy and postmortem tissues were preserved in fresh 4% deionized paraformaldehyde, embedded in paraffin wax, and sectioned to 5 μm. The sections were deparaffinized, rehydrated in Tris-buffered saline (TBS; 0.1 M Tris [pH 7.5], 0.1 M NaCl), and digested with proteinase K (20-40 μg/ml, 37° C., for 30-50 min; Sigma). For PCR-ISH detection of HIV-2 gag RNA, tissue sections were rehydrated, washed in DEPC water, and treated overnight at 37° C. in a RNase-free DNase-1 solution (Boehringer Mannheim), as described previously (Brodie, S. J. et al., Epizootic hemorrhagic disease. Analysis of tissues by amplification and in situ hybridization reveals widespread orbivirus infection at low copy number, J Virol 72:3863-3871 [1998a]; Brodie, Si. et al., The effects of pharmacological and lentivirus-induced immunosuppression on orbivirus pathogenesis: Assessment of blood monocytes and tissues by in situ hybridization and reverse transcription in situ PCR, J Virol 72, 5599-5609 [1998]). The sections were then incubated for 2 min at 70° C., followed by 50 min at 42° C. with a mixture of specific SIV antisense primer (SIV3Q2) and MuLV reverse transcriptase (RT-PCR kit; Perkin Elmer, Norwalk, Conn.). The gag cDNA or native gag DNA was then reacted with 50 μl of a PCR solution containing 50 pM of the HIV-2 gag-specific primers

(SEQ ID NO: 4)
HIV-2[5Qii2], 5′-TTGGATTGGCAGAGAGCCTGTTGGGAT;
and
(SEQ ID NO: 5)
HIV-2[3Qi2], 5′-TACCCAGGCATTTAAGGTTCGGG

and taq polymerase (0.15 U/μl). Primer sequences and thermocycling conditions were as described previously. (Rey-Cuille et al. [1994]; Brodie et al. [1999b] and [2000b]; Berrey, M. M. et al., Treatment of primary human immunodeficiency virus type 1 infection with potent antiretroviral therapy reduces frequency of reprogression to AIDS, J. Infect Dis 83:1466-1475 [2001]). The PCR amplification product was detected by ISH using a cocktail of three HIV-2 gag-specific oligonucleotides labeled with digoxigenin (DIG) or fluorescein (FAM)-11-dUTP (Boehringer Mannheim, Indianapolis, Ind.); all were in sense orientation, and internal to the following PCR primer binding sequences

(SEQ ID NO: 6)
HIV-2[3KDi], 5′-AATACCGTCTGCGTCATCTTTTGCC;
(SEQ ID NO: 7)
HIV-2[KDii], 5′-AGCACAGCGACATCTAGCAGCGGACACAG;
and
(SEQ ID NO: 8)
HIV-2[KDiii], 5′-AGCCGCCTAGCTTATCCAGTGCAGCAA.

A 0.8-kb riboprobe was developed for HIV-2/SIV gag, as was previously done for HIV-1, SIV, and other animal lentiviruses (Brodie, S. J. et al., Ovine lentivirus expression and disease: virus replication, but not entry, is restricted to macrophages of specific tissues, Am J Pathol 146, 1-13 [1995]; Brodie, S. J. et al., Macrophage function in simian AIDS: Killing defects in vivo are independent of macrophage infection, associated with alterations in Th phenotype, and are reversible with IFN-γ, J Immunol 153, 5790-5801 [1994]; Brodie, S. J. et al. [1998a]; Brodie et al. [1999a-b]; Brodie et al. [2000b]). The riboprobe was used to localize cells harboring HIV-2 gag RNA and to estimate intracellular viral copies.

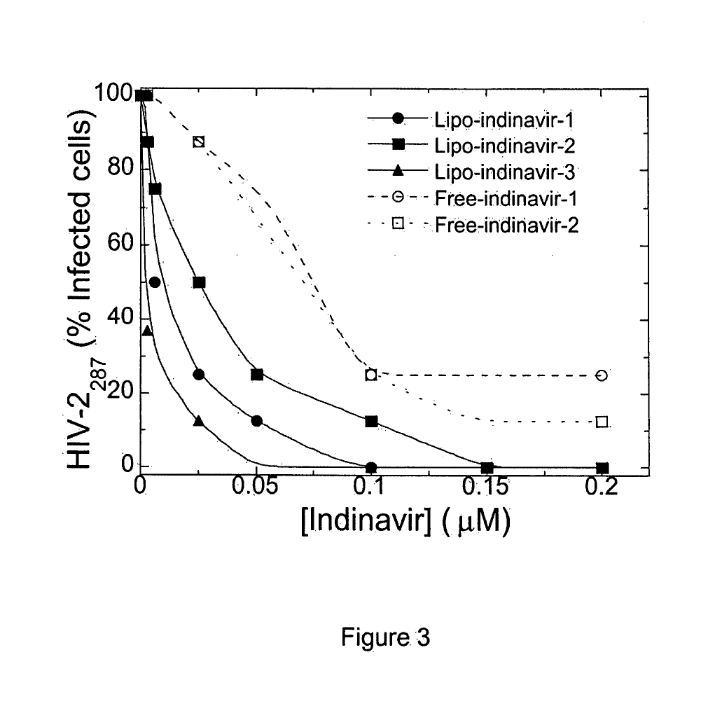

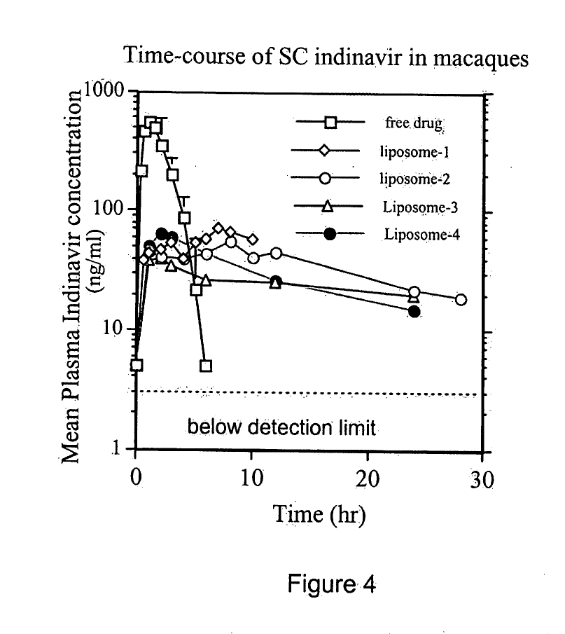

PCR-ISH and ISH were used to localize latent and low copies of HIV in a variety of tissues and cells (e.g., FIGS. 3 and 4). When combined with immunohistochemistry, the phenotype of the cell(s) harboring rare viral targets can be identified. Using these combined techniques, HIV-2 DNA and RNA can be localized to specific cell types based on morphology and expression of specific cell surface markers, including CD21⁺ and S100⁺ dendritic cells (Brodie et al. [1999a]; and Brodie, S. J., Nonlymphoid reservoirs of HIV-1 replication in children with chronic-progressive disease, J Leukoc Biol 68, 351-359 [2000e]), macrophages by markers to CD64 and CD68 antigens (Brodie et al. [1994], [1995], and [1999a]), and memory and naive T lymphocytes by detection of CD45RO⁺/CD62L⁻ and CD45RA⁺/CD62L⁺ isoforms (Brodie et al. [1999a-b], and Brodie et al. [2000a-b]).

Tissue controls for these assays consisted of HIV-2-infected and uninfected CEM-174 cells (e.g., FIG. 3) and vaginal and cervical tissues from retrovirus-negative animals. PCR and hybridization controls are the same as described previously (Brodie et al. [1998a]; Brodie, S. J. et al. [1998b]; Brodie et al. [1999a-b], and [2000b]) and included amplification in the absence of taq polymerase or specific primers, hybridization with nonsense probes, and incubation with irrelevant isotype-specific antibody. To validate the PCR, test and control samples were prepared and amplified simultaneously with reaction mixtures either containing or lacking taq polymerase and specific primers. The presence of HIV-2 RNA and DNA was indicated by a purple cell-associated precipitate (DIG label) or by green fluorescence (FAM label). Images from the representative low-power microscopic fields were transmitted to a computer equipped with a digital imaging board (Brodie et al. [1999a]) and the proportion of virus-infected cells determined. Intracellular viral copies were estimated based on total cellular fluorescence.

Isolation and characterization of virus-infected cells from lymph nodes: Activated (CD45RO⁺/CD62L⁻/HLA-DR⁺, plus CD25⁺, CD38⁺, CD69⁺, CD71⁺, cyclin A⁺, and/or Ki67⁺), quiescent (CD45RO⁺/CD62L⁻/HLA-DR⁻, plus CD25⁻, CD38⁻, CD69⁻, CD71⁻, cyclin A⁻, and Ki67⁻), and naïve (CD45RA⁺/CD62L⁺/HLA-DR7CD25⁻/CD38⁻/CD69⁻/CD71⁻) CD4⁺T cells were separated from PBMC and from LNMC using negative selection and magnetic bead removal, combined with fluorescent activated cell sorting (Brodie et al. [1994] and [2000b]). Briefly, mononuclear cells were suspended in RPM 1640, followed by a 1-h incubation (37° C. and 5% CO₂) in fibronectin-coated flasks (20 μg/ml) to remove adherent cells. The nonadherent lymphocyte-enriched population were labeled with mAbs to CD8, CD14, CD16, CD19, CD20, and CD21 to enrich for CD4⁺T-cells and with mAbs to HLA-DR, CD25 (IL-2R), CD38, CD69, CD71, cyclin A, and Ki67 to remove activated and/or proliferating cells. The cells were incubated with monoclonal antibodies (mAbs) for 30 min at 4° C., washed, and then reacted with secondary mAb conjugated to magnetic microspheres (Dynal, Great Neck, N.Y.) in a bead:cell ratio of 4:1 and incubated at 4° C. for 30 min. Rosetted cells were collected by magnetic particle isolation leaving the highly-enriched cell population. The enriched cells were further purified by fluorescent-activated cell sorting using mAbs specific to the lymphocyte subsets CD45RO (memory T cells) or CD45RA/CD62L (naïve T cells). By combining these techniques, >99% of purified cells expressed the specific cell surface markers for the T cell populations defined as activated, quiescent, and naïve. All of the proposed cell surface markers (mAbs) have been used successfully in humans and with varying degree of success in primate cells.

Lymphocyte subset analysis: Fluorescent labeled monoclonal antibodies to lymphocyte surface markers were used to quantitate populations of T cells (CD2+), helper T cells (CD4⁺), suppressor T cells (CD8⁺), and B cells (CD20⁺) in maternal (1 ml) and fetal (150 μl) blood using procedures previously described (Ho R J, Agy M B, Morton W R, et al. Development of a chronically catheterized maternal-fetal macaque model to study in utero mother-to-fetus HIV transmission: a preliminary report. J Med Primatol 1996;25 (3):218-24).

Quantification of HIV-2 RNA in subsets of phenotypically distinct T cells: In-situ hybridization was performed in parallel with immunocytochemistry in cell suspension to identify specific CD4⁺ T-cell subsets that allowed HIV-2 gag-pol transcription. Briefly, mAbs specific to cell-surface and intracytoplasmic proteins were applied in combination with HIV-2 RNA-specific oligonucleotide probes to assess HIV-1 transcriptional activity in subsets of CD4⁺ T lymphocytes (CD45RA or CD45RO) in differing states of activation (CD25, CD38, CD69, CD71, and HLA-DR) and stages of the cell cycle (Ki67 and cyclin A) using a flow cytometry-based detection strategy, as described previously for HIV-1 (Brodie [1999b], [2000a-b]). Mononuclear cells were labeled with fluorochrome (PE, cychrome, PC5, and/or ECD)-conjugated mAbs (PharMingen, San Diego, Calif.) specific to the cellular antigens described above and then fixed and permeabilized with Permeafix. The cells were then hybridized with a cocktail of fluorescein-labeled oligonucleotide probes spanning open reading frames of HIV-2₂₈₇ gag-pol and analyzed by flow cytometry. By evaluating ACH-2 cells containing one integrated copy of HIV-1 DNA and fixed at consecutive time-points following PMA stimulation of viral transcription, the limit of detection of HIV-1 gag-pol RNA within a single positive cell was ≦10 copies (Brodie et al. [1999b]).

Scanning cytometry (LSC): The LSC procedure provides data equivalent to the flow cytometry technique adapted for microscopic slide analysis. The LSC measures four-color fluorescence and light scatter and records the position and time of measurement of each cell. The LSC calculates total cellular fluorescence and can be used to determine a mean signal (viral) copy number within individual cells. It is faster than an image analyzer and shows better detail than a flow cytometer. LSC has been used to differentiate levels of signal intensity in single cells (e.g., Brodie et al. [2000a]).

Liquid chromatography-mass spectroscopy (LC-MS) assays to detect drug levels in blood and tissues: A liquid chromatography-mass spectroscopy (LC-MS) assay was used to detect indinavir in plasma and tissue (homogenate) samples using cyheptamide as internal control prior to extraction with CH₂Cl₂, by methods previously described. (e.g., Quian, M. et al., Metabolism of 3′-azido-3′ deoxythymidine (AZT) in human placental trophoblasts and Hofbauer cells, Biochemical Pharmacology 48, 383-389 [1994a]; Quian, M. et al., Comparison of intracellular metabolism of AZT in human and primate peripheral blood mononuclear cells, Antimicrobial Agents and Chemotherapy 38, 2398-2403 [1994b]).

One ml of macaque plasma or 1 mg of lymphoid tissues homogenized in lml buffer was extracted with CH₂Cl₂ at pH 8.0 according to Chen et al. (Chen I W, Vastag K J, Lin J H. High-performance liquid chromatographic determination of a potent and selective HIV protease inhibitor (L-735,524) in rat, dog and monkey plasma. J Chromatogr B Biomed Appl 1995; 672 (1):111-7), in the presence of 1 μg cyheptamide as an internal control. After extraction with CH₂Cl₂, the contaminants are removed with a silica column (1×4 cm), and the indinavir eluted in 2-propanol: CH₂Cl₂ (1:4 v/v) and dried under N₂. These samples were resuspended in 100 μL, and a 10 μL sample was injected onto an RX-C8 column (21 mm×15 cm column) and eluted isocratically with a mobile phase containing 20 mM ammonium acetate in acetonitrile:water (65:35, v/v) running at 0.38 ml/min. After atmospheric pressure ionization under an electrospray mode, the analytes were detected using selected-ion monitoring (SIM) at m/z 614.7-615.7 amu to detect indinavir. Under these conditions, the detection limit was 100 pg, which made it possible to measure drug levels, extract RNA and DNA, isolate cells for detailed analyses of vRNA and vDNA-infected cells in lymph nodes, and fix the tissue for pathological analyses from limited sample size.

Histological analysis of HIV-infected cells: Histologic sections of lymph node were examined using in situ hybridization for HIV-2 gag RNA. Biopsy and postmortem tissues were preserved in fresh 4% deionized paraformaldehyde, embedded in paraffin wax, and sectioned to 5 μm. The sections were deparaffinized, rehydrated in Tris-buffered saline (TBS; 0.1 M Tris [pH 7.5], 0.1 M NaCl), digested with proteinase K (20-40 μg/ml, 37° C., for 30-50 min; Sigma), and treated overnight at 37° C. in a RNase-free DNase-1 solution (Boehringer Mannheim), as described previously. (Brodie S B, Keller M J, Ewenstein B M, et al. Variation in incidence of indinavir-associated nephrolithiasis among HIV-positive patients. Aids 1998;12 (18):2433-7; Diamond C, Brodie S J, Krieger J N, et al. Human herpesvirus 8 in the prostate glands of men with Kaposi's sarcoma. J Virol 1998;72 (7):6223-7).

The sections were then incubated for 5 min at 70° C. followed by 4 h at 42° C. with antisense RNA probes directed either to SW (strain mac251; Zhang Z Q, Schuler T, Cavert W, et al. Reversibility of the pathological changes in the follicular dendritic cell network with treatment of HIV-1 infection. Proc Nati Acad Sci U S A 1999;96 (9):5169-72) or HIV-2 gag-pol (stain 287; gag 2639-1080, pol 3473-2306; Galabru J, Rey-Cuille M A, Hovanessian A G. Nucleotide sequence of the HIV-2 EHO genome, a divergent HIV-2 isolate. AIDS Res Hum Retroviruses 1995;11 [7]:873-4). The RNA probes were constructed by cloning cDNA into transcription vectors under the control of T7 and SP6 RNA polymerase promoters (pGEM, Promega; Madison, Wis.). Constructs were linearized and used as templates for in vitro transcription to which [³⁵S]-UTP (Amersham Corp., Arlington Heights, Ill.) was incorporated (˜2×10⁶ dpm/μg). After hybridization, the slides were washed in 5× standard saline citrate (SSC), 10 mM dithiothreitol (DTT) at 42° C., 2× SSC, 10 mM DTT, 50% formamide at 60° C., and a 2× RWS buffer (0.1 M tris-HCl (pH 7.5), 0.4 M NaCl, 50 mM EDTA) before digestion with ribonuclease A (25 μg/ml) and T₁ (25 units/ml) in 1× RWS. After washing in RWS, 2× SSC, and 0.1× SSC, sections were dehydrated in graded ethanol containing 0.3 M ammonium acetate and then air-dried and dipped in Kodak NTB-2 emulsion, exposed at 4° C., developed, and lightly counterstained. HIV-2 RNA was detected after autoradiographic exposures of 24 and 96 h.

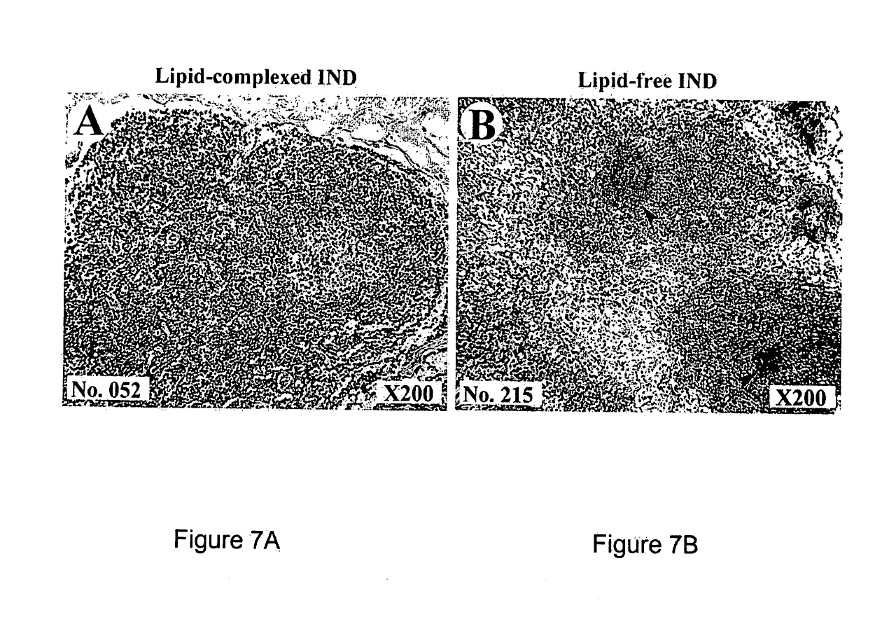

The presence of viral RNA was indicated by deposition of silver grains on top of cells or as aggregates within lymph node germinal centers (FIG. 7) at a frequency statistically greater than background, as determined using Image-Pro® Plus software (Media Cybernetics, Silver Spring, Md.). Nonspecific background was determined in two ways with equivalent results. Silver grains were counted over 100 germinal center cells in tissue sections from mock-inoculated uninfected animals after hybridization to the HIV-2 or SIV antisense probe, or over cells from the tissues of infected monkeys after hybridization to noncomplimentary ‘sense’ RNA probes. In both cases, the average background was 0.3 grains per cell for the 24 h exposure. Additional controls consisted of tissue sections, with and without protease treatment, and antisense RNA from the visna virus gag gene. (Brodie S J, de la Rosa C, Howe J O, et al. Pediatric AIDS-associated lymphocytic interstitial pneumonia and pulmonary arterio-occlusive disease: role of VCAM-1/VLA-4 adhesion pathway and human herpesviruses. Am J Pathol 1999;154 (5):1453-64; Brodie S J, Pearson L D, Zink M C, et al. Ovine lentivirus expression and disease. Virus replication, but not entry, is restricted to macrophages of specific tissues. Am J Pathol 1995;146 (1):250-63).

The Poisson probability that x number of grains differs from a background average of m is P=(m^{x}×e^{-m})/x. For a cell with grains over background, the probability that the cell is infected is >0.99. Using previously validated back-calculation methods (Haase A T, Henry K, Zupancic M, et al. Quantitative image analysis of HIV-1 infection in lymphoid tissue. Science 1996;274 (5289):985-9; Haase A T, Stowring L, Harris J D, et al. Visna DNA synthesis and the tempo of infection in vitro. Virology 1982;119 (2):399-410; Zhang Z Q, Schuler T, Cavert W, et al. Reversibility of the pathological changes in the follicular dendritic cell network with treatment of HIV-1 infection. Proc Natl Acad Sci U S A 1999;96 (9):5169-72). In the study described herein, one silver grain approximated 2 copies of HIV-2 RNA. The amount of viral RNA within germinal centers was also calculated. Silver grain counts were averaged per six consecutive LN germinal centers (50× microscopic fields) at the 24 h exposure. Similar results were achieved with both SIV and HIV-2 RNA probes.

### Example 2

### The Effect of pH on the Ability of Drugs to Associate to Lipid Bilayer

FIG. 1A illustrates the pH-dependent incorporation of indinavir within lipid-associated complexes. With lipids containing phosphatidyl choline (egg): cholesterol (3:1 molar ratio) and lipid to indinavir (5:1 molar ratio), small lipid particles were prepared with phosphate-buffered saline at indicated pH value. They were sonicated to achieve 55±5 nm in diameter. Subsequently, the % lipid-association was determined by separating free from lipid-associated drug by size-exclusion column chromatography. Data expressed were means of duplicate preparations for indicated pH value.

It has been reported that changing the pH of indinavir from pH 3 to pH 7 results in a 1000-fold decrease in its aqueous solubility as provided in Table 1A (Lin et al., pH-dependent oral absorption of L-735,524, a potent HIT/protease inhibitor, in rats and dogs, Drug Metab Dispos 23:730-735 [1995]). The effect of pH on the ability of indinavir to incorporate or associate with lipids in forming lipid-drug complexes was determined. At a lipid-to-drug ratio of 5:1 (m/m), practically all (85-95%) of the drug in the preparation was found to be associated with liposome at pH 7.4, as illustrated in FIG. 1A. At lower pH values (e.g., pH 4), where aqueous solubility of the drug was higher, a much lower proportion (<30%) of the drug was incorporated into the lipid bilayer of liposomes. Since physiological pH is 7.4, and because biological fluids are highly buffered, lipid-associated drugs are expected to remain stable under these conditions. The lipid-indinavir complexes formed and maintained at pH 7.4 were used for the subsequent pharmacokinetic studies. Under this set of conditions, lipid-associated indinavir exhibited a diameter of 69±7 nm as provided in Table 1B.

FIG. 1B illustrates the pH-dependent release of indinavir from lipid-associated complexes in vivo. The pH dependence on the lipid and drug association is also consistent with the observation that the lipid-associated drug can be released in a pH-dependent manner. Because of the stringent active-site-binding requirement and pharmokinetic profiles, most of the clinically used anti-HIV protease inhibitors, including saquinavir, ritonavir, indinavir, lopinavir, and nelfinavir, exhibit a profile with similar lipophilicity, which is also pH-dependent. See Table 1B. We found that saquinavir and nelfinavir exhibit a high degree of lipid association, similar to indinavir as provided in Table 1B.

TABLE 1A
pH-dependent Effects of Indinavir
Solubility and Lipophilicity

| | Solubility | |
|---|---|---|
| pH | in aqueous | LogP (lipophilicity) |
| 3.5 | 50 mg/ml | 0 |
| 5 | 0.1 mg/ml | 1.8 |
| 7 | 0.01 mg/ml | 3 |

From Lin et al., 1995 Drug Metabolism and Disposition 23:730-5

TABLE 1B
The Characterization of Anti-HIV drugs Associated with Lipid Membranes

| | | Degree of Lipid | Size in |
|---|---|---|---|
| | | Association | Diameter |
| Anti-HIV Drug | Class of Drug | (% total) | (nm) |
| Indinavir | Protease inhibitor | 97.5 ± 2.5 | 69 ± 7 |
| Saquinavir | Protease inhibitor | 98.8 ± 7.5 | 159 ± 36 |
| Nelfinavir | Protease inhibitor | 100.5 ± 9.8 | 123 ± 16 |
| Viread ® | RT inhibitor^{a} | 4.2 ± 0.7 | 60 ± 10 |

^{a}Reverse transcriptase inhibitor, adenosine nucleotide analog.

TABLE 1C
Effects of Protease inhibitor combination on efficiency of
drug incorporation in lipid-drug nanoparticles

| Drug in | | | | | |
|---|---|---|---|---|---|
| DSPC:mPE- | | | | | |
| DSPE | % Associated with lipid-drug nanoparticles | | | | |
| formulatio | Indinavir | Lopinavir | Ritonavir | Nelfinavir | Saquinavir |
| Indinavir | 97.5 ± 2.50 | — | — | — | — |
| Nelfinavir | — | — | — | 100 ± 9.8 | — |
| Saquinavir | — | — | — | — | 98.9 ± 7.5 |
| Lopinavir | — | 100 ± 0.9 | — | — | — |
| Ritonavir | — | — | 100 ± 1.0 | — | — |
| Lopinavir + | — | 94 ± 6.2 | 96 ± 12 | — | — |
| Ritonavir | | | | | |
| Indinavir + | 93.0 ± 0.18 | — | — | 94.2 ± 1.9 | — |
| Nelfinavir | | | | | |

### Example 3

### Comparison of Indinavir Concentrations in Human Lymph Node and Peripheral Blood Mononuclear Cells (PBMCs)

To determine the indinavir concentrations in mononuclear cells of lymph nodes (LNMC) in relation to that of blood (periphery), we employed a highly sensitive liquid chromatography-mass spectroscopy (LC-MS) assay to estimate indinavir content in 5×10⁵ of each type of cell. The matched peripheral blood mononuclear cells (PBMC) and lymph node mononuclear cells (LNMC) data (collected from blood and lymph node respectively) from 3 HIV⁺ patients treated by HAART, including indinavir, indicated that the indinavir concentrations in LNMC were much lower than in PBMC (Table 2). These data indicated that indinavir concentration in lymph node mononuclear cells was much lower than in their blood counterpart, with the ratio ranging from 0.23 to 0.35 (i.e., less than unity or 1). Assuming that each mononuclear cell has a volume of 4×10⁻⁹ ml³ (a value estimated for mammalian cells in Alberts et al., Macromolecules; structure, shape, and information. In “Molecular Biology of the Cell”, 3rd edition, Garland Publishing, Inc., N.Y., pp 89-90 [1994]), the intracellular indinavir concentration for Patient JS1166's PBMC was calculated as 0.86 μg/ml, a value similar to the plasma concentration (0.616 μg/ml). The similar indinavir concentration observed in plasma and PBMC is consistent with the data of Lin et al. (Lin et al., Species differences in the pharmacokinetics and metabolism of indinavir, a potent human immunodeficiency virus protease inhibitor, Drug Metab Dispos 24:1111-20 [1996]), demonstrating that indinavir in plasma can equilibrate readily with erythrocytes in blood. More importantly, these data indicate that indinavir concentrations in lymph nodes, particularly mononuclear cells, are much lower than in plasma and blood cells. Therefore, enhanced drug delivery to lymphoid organs, particularly to lymph nodes, should significantly improve control of viral replication in lymphoid tissues.

TABLE 2
Indinavir Concentration in Mononuclear Cells of Peripheral
Blood, Lymph Nodes, and Plasma*

| | | PBMC | LNMC | LNMC |
|---|---|---|---|---|
| | Plasma | (ng/5 × 10⁵ | (ng/5 × 10⁵ | ratio |
| Patient ID | (μg/ml) | cells) | cells) | PBMC |
| JS1166 | 0.616 | 1.72 | 0.60 | 0.35 |
| JA1216 | NA | 1.88 | 0.53 | 0.28 |
| SS1196 | NA | 0.53 | 0.124 | 0.23 |

*Lymph node mononuclear cells were isolated from lymph node biopsy collected at the same time when the blood samples were collected to isolate plasma and PBMC from the same patients. Cellular indinavir concentrations were determined by LC-MS using indinavir extracted from 50,000 cells. Data expressed were mean of duplicate extractions of each sample.
NA: no sample available.

### Example 4

### Characterization of a Highly Pathogenic Strain of HIV-2 Infection in Macaques

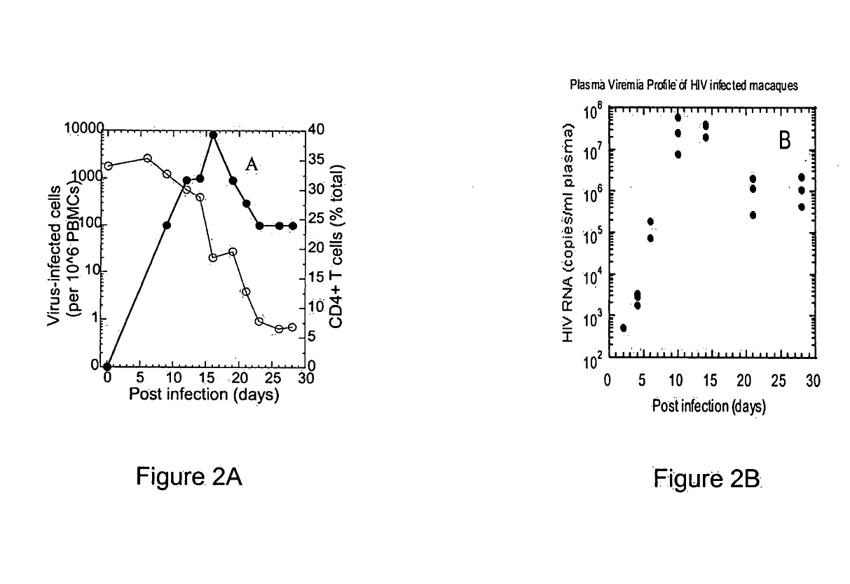

In more than 20 HIV-2₂₈₇-infected pregnant macaques (Macaca nemestrina), a prominent viremia phase was observed that peaked within 2 weeks, detected as corresponding peaks in virus-infected cells in blood and free virus (quantitated by QC-RNA-PCR) in plasma, and a subsequent rapid CD4⁺-T cell decline within 3 weeks post-infection (Ho et al., Development of a chronically catheterized maternal-fetal macaque model to study in utero mother-to-fetus HIV transmission—a preliminary report. An invited article, J Medical Primatol 25:218-24 [1996]). A typical time course of disease progression following HIV-2₂₈₇ is graphically presented in FIG. 2A in terms of virus load and CD4⁺ T-cell depletion. In FIG. 2A, a representative macaque (pregnant macaque at 140 d gestation) was inoculated with 10 TC_{ID50} of HIV-2 (IV) and virus-infected PBMC (shaded points), and CD4⁺-T cells (unshaded points) were monitored every other day until delivery of the infant by C-section.

FIG. 2B illustrates the analysis of plasma for a viral RNA profile of 27 macaques that were infected with 50 TC_{ID50} HIV-2₂₈₇. Each data point represent a sample collected from each animal. FIG. 2B indicates that virus was detectable in the plasma at day 4 and reached a peak value of approximately 5×10⁷ copies/ml between days 10 and 14 after infection. The viral load after the acute phase of infection (“viral set-point”) was reached at day 21 and remained detectable consistently at approximately 10⁶ copies/ml thereafter. These results also demonstrate the rapid progression and highly reproducible nature of HIV-2₂₈₇ infection in pig-tailed macaques. These properties are highly desirable for antiviral drug studies, because a much shorter time frame is needed with perhaps greater statistical power to detect therapeutic effects.

To determine the time course of virus levels in lymph nodes, 27 macaques were infected intravenously with HIV-2₂₈₇ at a dose of 50 TC_{ID50}. Three animals were sacrificed on each of the following days after infection: 0.5, 1, 2, 4, 6, 10, 14, 21, 28-30. The following tissues were collected: peripheral blood mononuclear cells, bone marrow, spleen, ileocecal lymph nodes, inguinal lymph nodes, axillary lymph nodes, mesenteric lymph nodes, deep pelvic lymph nodes, submandibular lymph nodes, and thymus. Viral load in each of these tissues was determined by quantitative co-culture with PHA-activated CD8⁺ cell-depleted human PBMC. The results indicated that productively infected cells were first detectable in all tissues between 4-6 days after infection at a level of 1-100 infectious cells per million. The viral load peaked between days 10-14 reaching a level of approximately 10³-10⁴ infected cells per million (i.e., 0.1-1% of cells were productively infected). This level decreased after acute infection, but remained detectable at 10-1000 infected cells per million between 21-30 days after infection.

### Example 5

### The Effects of Lipid Association on the Ability of Indinavir to Inhibit HIV-2₂₈₇ Replication

FIG. 3 illustrates the concentration-dependent inhibition of HIV-₂₈₇ replication by free (not lipid-associated) and lipid-associated indinavir. HIV-2₂₈₇-infected CEM-174 cells (0.01 multiplicity of infection [MOI]) were incubated with the indicated concentrations of indinavir, in free (open symbols) or lipid-associated (closed symbols) formulation, and drug effects on virus replication are expressed as mean % infected cells of quadruplicate samples that were assayed for the presence of p27 core antigen of HIV-2. Under these conditions, all the control samples without drugs were positive for viral replication. The effective concentrations that produce half the maximum anti-HIV activity (EC₅₀) were determined based on non-linear regression of each set of data, representing the frequency of replication.

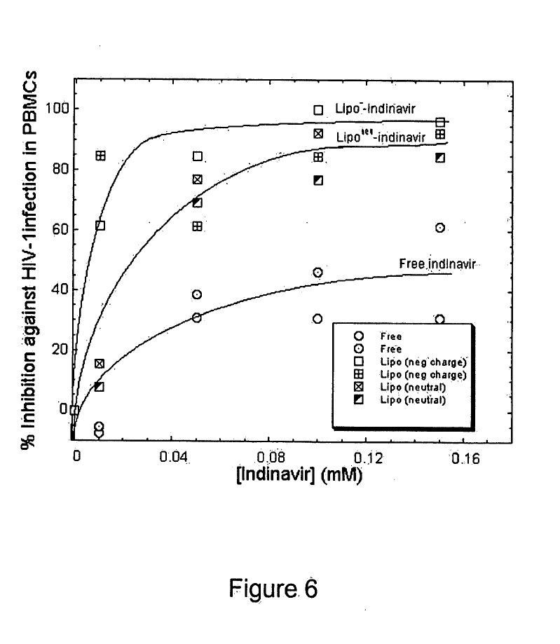

To determine anti-HIV activity of lipid-associated indinavir, 5×10⁵ CEM-174 cells were infected with 5×10³ TC_{ID50} (multiplicity of infection, MOI=0.01) HIV-2₂₈₇ in 2 ml RPMI 1640 tissue culture medium containing 1% fetal calf serum for 1 hr at 37° C. After unabsorbed virus was removed by washing the cells with medium, 100 μl of suspensions containing 10⁴ infected cells were transferred to flat-bottom, 96-well microliter plates containing 100 μl of serially diluted (0-15 μM) indinavir, either in free or liposome-associated formulations. After incubating the cells at 37° C. in RPMI 1640 containing 10% fetal calf serum for 4 days (optimum detection time), the presence of virus-infected cells was determined visually by the presence of syncytia and was subsequently confirmed by ELISA detection of the presence of HIV-2 antigen. Experiments were repeated on at least two different days with each determination done in quadruplicate samples, and the data presented in FIG. 3 are the mean % virus-infected cells. Regression analysis estimated the EC₅₀ (50% effective inhibitory concentration) value for lipid-associated indinavir to be 0.01-0.025 μM, and 0.05-0.08 μM for free indinavir. These data imply that lipid-associated indinavir is about 3- to 6-fold more potent than free drug in inhibiting HIV-2₂₈₇ replication. A similar degree of enhancement was recorded for HIV-1_{LAI}infected human PBMC. FIG. 6 shows the concentration-dependent inhibition of HIV-1_{LAI} replication by the free and lipid-associated indinavir.

### Example 6

### A Plasma Time Course Profile of Free versus Lipid-Associated Indinavir in Macaques

FIG. 4 illustrates a time course for plasma concentration of indinavir following the subcutaneous administration of lipid-associated and non-lipid-associated indinavir within macaques. Young adult male macaques were given either free or liposome-formulated indinavir at 10 mg/kg body mass per dose, and plasma drug concentrations were determined by HPLC assay. Data expressed were means ±SD for animals injected with free (open squares, n=4) and lipid-associated indinavir (referred to as symbols; liposome-1, liposome-2, liposome-3, liposome-4). FIG. 4. Young adult (5-6 kg body mass) macaques that were administered subcutaneously (SC) with either free or lipid-associated indinavir (SC) at 10 mg/kg body mass per single dose. Free indinivir, solubilized in DMSO and phosphate buffer suspension, produced a plasma drug concentration peak at about 0.5-1 hr, and rapidly cleared the drug to below the limit of detection in plasma by 6 hr (FIG. 4). In contrast, lipid-associated indinavir produced a peak plasma concentration about 10-fold lower than free drug, and sustained this plasma level beyond 10 hr due to sustained drug release from lymph nodes. When a second dose was given after a 30-day washout period, a significant amount of drug (>20 ng/ml) remained in plasma beyond 24 hr (FIG. 4; profile of liposome-1 and -2). Animals labeled as liposome-3 (M98165) and -4 (J98328) were previously infected with HIV-2₂₈₇ and, hence, allowed collection of the visceral lymph nodes for drug analysis. The data are presented in Table 3 provided in Example 7 below.

### Example 7

### The Effect of Lipid-Drug Complexes on Enhanced Accumulation of Indinavir in Lymph Nodes

In some experiments, lipid-associated indinavir (10 mg/kg body mass) was administered to two additional HIV-2₂₈₇-infected macaques, and inguinal lymph nodes were harvested at 6, 24 and 16 or 28 hrs. Drug concentration was measured in blood as well as lymph nodes. Time-course plasma drug concentrations of these two animals are presented in FIG. 4 (liposome-3 and -4).

In contrast, in two macaques that were administered lipid-associated indinavir, the lymph-node-to-plasma ratio ranged from 2.5- to 22.7-fold between 6 and 28 hrs post-administration provided in Table 3. The variability in drug accumulation between lymph nodes may be due to the limited flow and diffusion rates of the lipid-drug particles within the lymphatic systems. The variability can be reduced by administration of the lipid-indinavir in multiple sites or repeated dosing schedule. Even at 24 to 28 hrs, 20-30 ng/ml of indinavir was available in blood. Given the in vitro ED₅₀ of indinavir, 0.001-0.025 μM or 7-17 ng/ml against HIV-2₂₈₇ for lipid associated form, and 42-56 ng/ml (0.06-0.08 μM) for free drug, these values are within its acceptable, but low, therapeutic range. Hence, the dose of indinavir should be increased 2- to 4-fold (20-40 mg/kg body mass) to achieve higher plasma drug levels to produce maximum effect on virus load reduction.

Three HIV-2₂₈₇-infected macaques, administered 25 mg/kg body mass oral indinavir, showed minimal levels of drug presence in either axillary or inguinal lymph nodes provided in Table 4. In contrast, in animals administered with lipid-associated indinavir, we found that the lymph node-to-plasma ratio ranged from 2.5- to 22.7-fold between 6 and 28 hrs post-administration in two animals (Table 3). Data collected from 25 mg/kg body mass oral indinavir to HIV-infected macaques exhibited no detectable indinavir in plasma or lymph nodes beyond 8 hr; more importantly, at any given time point, lymph node-to-plasma drug ratios never exceeded unity (Table 4). Oral indinavir administration to rats by Lin et al. indicates that while [¹⁴C]-labeled indinavir rapidly distributed into the mesenteric lymph, it was cleared from the lymphatic system at a much faster rate than from plasma. (Lin et al., Species differences in the pharmacokinetics and metabolism of indinavir, a potent human immunodeficiency virus protease inhibitor, Drug Metab Dispos. 24:1111-20 [1996]). These data imply that lipid-associated indinavir provides enhanced lymph node accumulation of drug at levels that cannot be achieved by free drug administration; additionally, lipid association can produce sustained therapeutic levels in blood for a much longer duration.

Furthermore, these data also imply that lipid-drug complexes are sufficiently stable in vivo. If lipid-drug complexes were dissociated (to release free drug) at injection sites, free drug diffused or perfused to lymph nodes would produce much lower concentrations than those found in blood, and would never reach higher concentrations than in blood. In this case, lymph node to blood ratios would be around or less than one.

TABLE 3
Indinavir Accumulation in Selected Lymph Nodes of HIV-infected
Juvenile Macaques after Subcutaneous Administration of 10 mg/kg
Body Mass Lipid-associated Indinavir

| | | | Lymph | | |
|---|---|---|---|---|---|
| | | | node | Plasma | Lymph node |
| Animal | Lymph | Time | (indinavir) | (indinavir) | to |
| ID | node | (hr) | (ng/ml) | (ng/ml) | plasma ratio |
| M98165 | Inguinal | 6 | 1004.3 | 44.2 | 22.7 |
| | Inguinal | 28 | 109.2 | 31.8 | 3.4 |
| | Mesenteric | 28 | 158.8 | 31.8 | 5.0 |
| | Ileocecal | 28 | 1035.2 | 31.8 | 32.5 |
| | Axillary | 28 | 78.3 | 31.8 | 2.5 |
| J98328 | Inguinal | 24 | 147.6 | 20.7 | 7.1 |
| | Inguinal | 26 | 130.4 | 20.8 | 6.3 |
| | Mesenteric | 26 | 338.3 | 20.8 | 16.3 |
| | Ileocecal | 26 | 145.8 | 20.8 | 7.0 |
| | Axillary | 26 | 51.6 | 20.8 | 2.5 |

TABLE 4
Indinavir Accumulation in Select Lymph Nodes of HIV-infected
Macaques at Indicated Time Point after Oral Administration of
Free Indinavir (25 mg/kg body mass) in Solution

| | | | Lymph | | |
|---|---|---|---|---|---|
| | | | node | | |
| | | | (indinavir) | Plasma | Lymph node to |
| Animal | Lymph | Time | * | (indinavir) | plasma |
| ID | node | (hr) | (ng/ml) | (ng/ml) | ratio |
| 94079 | Inguinal | 3.25 | 0.283 | 9.8 | 0.03 |
| | Axillary | 3.25 | 0.128 | 9.8 | 0.01 |
| 94094 | Inguinal | 2 | 0.056 | 5.2 | 0.01 |
| | Axillary | 2 | 0.084 | 5.2 | 0.02 |
| 94096 | Inguinal | 3.75 | 0.481 | 39.8 | 0.01 |
| | Axillary | 3.75 | 0.141 | 39.8 | 3.5 × 10⁻³ |

### Example 8

### The Effect of Lipid-indinavir Complex on HIV-2₂₈₇ Infected Macaques

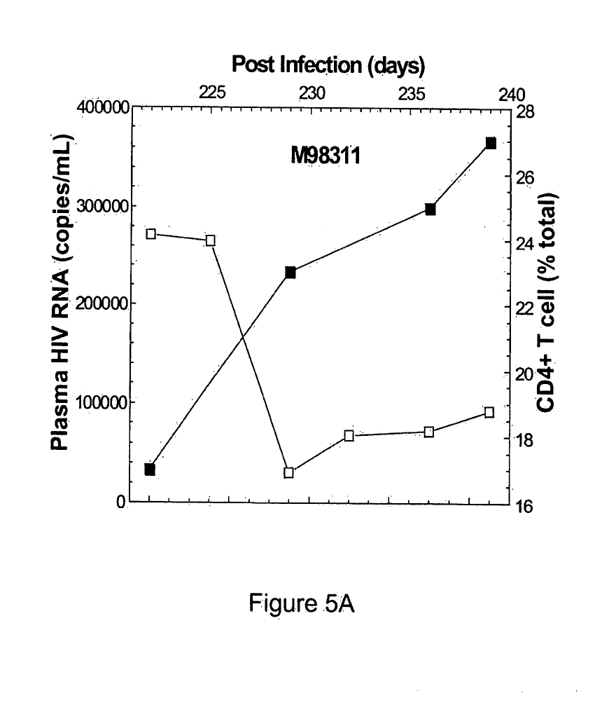

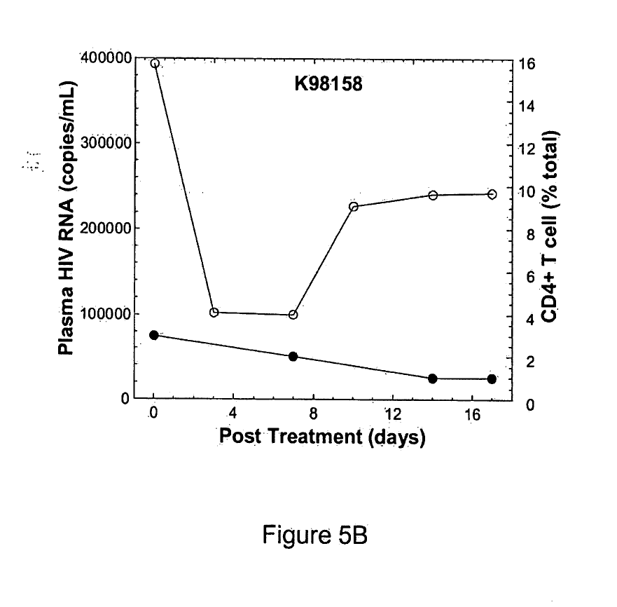

FIGS. 5A-B illustrates the changes in plasma virus load and the CD4⁺ T-cell profile monitored in two HIV-2₂₈₇-infected macaque at 25 weeks post-infection. FIG. 5A (macaque ID M98311) and FIG. 5B macaque (K98158) show the time-course of plasma viral RNA level (closed symbols) and CD4⁺ T-cell count (open symbols). Each macaque was injected subcutaneously with a single daily dose of 20 mg/kg body mass of lipid-indinavir on 10 days over a 14-day period. Two macaques infected with HIV-2₂₈₇ (about 30 weeks post-infection), exhibiting different degrees of disease progression, were treated with lipid-indinavir complexes over a 14-day period. At initiation of indinavir therapy, the CD4⁺ T-cell concentrations in one animal had declined below 100 (or 3% of total lymphocytes), and this animal did not reverse CD4⁺ T-cell decline in response to the drug therapy. This observation is consistent with that of human subjects under HAART where patients with CD4⁺ T-cells below 200 are less likely to respond to drug therapy. The second animal exhibiting CD4⁺ T-cells above 200 levels (at initiation of therapy) responded to the lipid-indinavir therapy. Analysis of virologic and T-cell responses indicated that even with the dose that was not optimized (10 single daily doses of 20 mg/kg body mass/day given over a 14-day period), lipid-indinavir significantly reduced the plasma virus load by day 6. The reduction in plasma virus load was reflected in CD4⁺ T-cell profile that rebounded by day 5, and sustained at this new level (>25%) even after cessation of the drug therapy at day 13.

About 20-fold higher indinavir concentration (1.2 μg/g in lymph nodes vs. 50 ng/ml in plasma at 14 hr) was achieved in axillary lymph nodes, distal to the lower scapular subcutaneous injection site at 13.3 hrs post-injection in the second animal. These data imply that subcutaneously administered lipid-indinavir complexes may distribute and accumulate in lymphoid tissues throughout systemic circulation and provide sustained lymph node as well as plasma indinavir levels. Also, the results confirm the data presented in Table 3. Sustained plasma drug levels were evident as shown by the continued presence of plasma indinavir (26 ng/ml) on day 17, more than 3 days (or 85 hrs) after the last dose of lipid-indinavir, was given to the second animal.

With four additional HIV-2₂₈₇-infected animals at various stages of disease progression, we compared the effects of lipid association on the ability of indinavir to alter the pathogenesis. Plasma cholesterol level was monitored to evaluate the effects of cholesterol given as a part of lipid formulation. As shown in Table 5, while these macaques were infected with varying doses of HIV-2₂₈₇ and exhibited different degrees of peak plasma virus load, animals treated with lipid-associated indinavir for two weeks showed an increase in CD4⁺ T cells, while the same pattern was not observed with animals treated with free indinavir. Furthermore, the additional dose of cholesterol given in the lipid-drug formulation did not alter the overall plasma cholesterol level.

TABLE 5
Effects of Indinavir Therapy on CD4⁺T cell and
Cholesterol Levels in HIV-2- Infected Macaques^{a}

| | | Peak viral | | | Plasma | |
|---|---|---|---|---|---|---|
| | Initial HIV | RNA | CD4⁺T cell | | Cholesterol | |
| | Inoculation | in Plasma | (per μL) | | (mg/dL) | |
| Macaques | Dose^{b} | (copies/mL)^{c} | Before^{d} | After^{d} | Before^{d} | After^{d} |
| Lipid-free indinavir treated | | | | | | |
| 215 | 1 | 3.2 × 10⁶ | 1946 | 1473 | 121 | 125 |
| 283 | 0.1 | 1.1 × 10⁵ | 1650 | 1051 | 193 | 188 |
| Lipid-associated indinavir treated | | | | | | |
| 052 | 1000 | 2.3 × 10⁷ | 242 | 527 | 175 | 169 |
| 225 | 0.1 | 5.3 × 10⁵ | 875 | 1707 | 124 | 136 |

^{a}Macaques, previously inoculated with HIV-2 287 at 33 weeks post infection, were treated with 22 mg/kg daily doses of indinavir for 14 doses. Animals 215 and 283 were subcutaneously treated with soluble indinavir formulation while 052 and 225 were treated with lipid-associated indinavir formulation. These animals had not been treated previously with any anti-HIV therapy.
^{b}Macaques were inoculated at indicated dose of HIV-2₂₈₇. All animals except 052 were inoculated by intravenous route. Macaque 052 was inoculated by intravaginal route as a part of a viral dose titration study. About a thousand-fold higher dose of virus is required, typically, to produce HIV infection in these animals.
^{c}Peak plasma viremia was observed within 2-3 weeks post viral inoculation and analyzed with an RT- QPCR and expressed as copies/mL.
^{d}The CD4⁺T-cell concentration and plasma cholesterol levels were measured before and after indinavir drug therapy.

### Example 9

### Lipid Association Enhances the Ability of Indinavir to Inhibit HIV-1 Replication in Human Peripheral Blood Mononuclear Cells

FIG. 6 shows the concentration-dependent inhibition of HIV-1_{LAV} replication by the free and lipid-associated indinavir. HIV-1-infected PBMCs were incubated with indicated concentrations of indinavir either in free (circles) or lipid-associated (squares) formulation, and drug effects on virus replication are expressed as mean % inhibition of duplicate samples that are assayed for the presence of HIV-1 p24 antigen. Under these conditions, all the control samples without drugs were positive for viral replication. The effective concentrations that produce half the maximum anti-HIV activity (EC₅₀) were determined based on non-linear regression of each set of data, representing the frequency of replication.

To evaluate the role of lipid formulation on indinavir's potency against HIV-1 replication, CD8 cells depleted, human peripheral blood mononuclear cells (PBMCs) (previously stimulated with PHA and IL-2, as described in Example 1, were infected with HIV-1_{LAV}. The 10⁴ HIV-1 infected PBMCs were exposed to 200 μl of serially diluted (0-15 μM) indinavir suspensions in free or liposome-associated formulations expressing either net positive or negative charge. Virus replication was assessed by measuring HIV-1 p24 antigen presence in the culture supernatant. Experiments were repeated on two different occasions with each determination done in duplicate, and the data presented are the mean % inhibition (FIG. 6). Regression analysis estimated the EC₅₀ value for lipid-associated indinavir to be 0.02-0.03 μM and >0.15 μM for free indinavir. Even at 15 μM, free indinavir did not exhibit 100% inhibition. These data implies that lipid-associated indinavir is more potent than free drug in inhibition of HIV-1 replication.

### Example 10

### Liposome-indinavir Complexes Accumulate in Lymphoid Tissues and Reduce HIV Viral Load in Infected Macaques

In additional experiments, data were collected from four HIV-2₂₈₇-infected macaques (Macaca nemestrina at 30 weeks post-infection), treated with 20 mg/kg/day subcutaneous lipid-indinavir complexes or free indinavir for 14 days. Similar to data presented in Table 3, about 20-fold higher indinavir concentration was achieved in axillary lymph nodes, distal to the lower scapular subcutaneous injection sites in lipid-indinavir-treated animals (at 13.3 hrs post-injection, data not shown). These results imply that lipid-indinavir complexes accumulate throughout lymphoid tissues. Sustained plasma drug levels were detectable even a few days after lipid-indinavir administration. At 3 days after cessation of lipid-inidnavir in the two animals, about 30 ng/ml plasma indinavir was detected. Drug levels in the two animals treated with free indinavir subsided below detectable levels by 4-5 hrs.

Virologic and T-cell analyses indicate that even with this unoptimized lipid-indinavir dose (14×[20 mg/kg body mass/day] dose given over 14 days) indinavir treatment had significantly reduced the plasma virus load by day 6. Animals treated with free indinavir did not exhibit a significant virus load reduction under these conditions. The reduction in plasma virus load of animals treated with lipid-indinavir was reflected in CD4⁺ T-cell profile that rebounded by day 5, and sustained at this new level (>25%) even after cessation of drug therapy (day 20). These data are similar to those presented in FIG. 5.

FIGS. 7A-B shows in situ hybridization analysis of lymph node sections in indinavir treated animals with a [³⁵S]-labeled HIV-2₂₅₇-specific probe. HIV-2-infected macaques were treated with formulations of 20 mg/kg body mass of lipid-indinavir complexes [lipid-complexed (IND)] (FIG. 7A, 052) and free indinavir [lipid-free IND] (FIG. 7B, 215) for 14 days, and lymph nodes were collected by necropsy on day 20. Only animals treated with lipid-free drug showed evidence of HIV-2 RNA in lymph node germinal centers (arrows). Analysis was performed on viral RNA expressing cells (by in-situ hybridization to detect viral RNA) in axillary and mesenteric lymph nodes of these four HIV-infected animals; two treated with lipid-indinavir complexes (animals 052 and 225) and two with lipid-free IND (macaques 215 and 283). Examination of axillary and mesenteric lymph nodes from the four HIV-2₂₈₇-infected animals, two treated with lipid-associated indinavir (animals 052 and 225) and two with lipid-free indinavir (animals 215 and 283) was performed. Representative photomicrographs of axillary lymph node sections hybridized with an [³⁵S]-labeled-HIV-2₂₈₇ RNA probe are shown in FIG. 7. Only the animals treated with free drug showed aggregates of HIV-RNA in lymph node germinal centers, the sites to which follicular dendritic cells are restricted. Both axillary and mesenteric lymph nodes were positive for HIV-2 RNA in these two animals provided in FIG. 7B. Macaque 215 had slightly higher concentrations of viral RNA in its lymph nodes (13,290±1,450 gag-pol RNA copies/50× field; FIG. 7B), compared to macaque 283 (8,134±890 gag-pol RNA copies/50× field). Free indinavir-treated animals showed only slightly less accumulation of viral RNA than the untreated HIV-infected control animals (9,222±1,100 gag-pal RNA copies/50× field; P>0.05). In contrast, lymph nodes from animals treated with lipid-indinavir showed much reduced viral RNA (FIG. 7A), with silver grain counts ranging from 337 to 1,280 (mean, 765±94 grains) per 50× microscopic field (P<0.001). In contrast, lymph node samples from animals treated with lipid-indinavir showed much reduced viral RNA by in situ hybridization provided in FIG. 7A.

Collectively, these data indicate that lipid-indinavir complexes are highly efficient in reducing the plasma virus load in vivo, and in reversing the CD4⁺ T-cell decline (due to natural course of HIV-2₂₈₇ infection). The indinavir delivered in lipid-indinavir complexes provided sustained and high drug concentrations in lymph nodes. The in-situ virus analysis of lymph nodes clearly indicates that treatment with lipid-indinavir complex, but not free indinavir treatment, had significantly reduced virus load in lymph nodes. Also, lipid-indinavir did not appear to influence lymph node structure, and therefore, this strategy may greatly reduce dose-limiting toxicity observed with systemic (plasma) exposure of high-dose indinavir. These data add significantly to the likelihood of success in achieving our goal to determine whether subcutaneous (SC) lipid-indinavir treatment is effective in reducing virus load in plasma as well as lymphoid tissues in HIV-infection and pathogenesis.

### Additional Discussion, Additional Experimental Results, and Summary

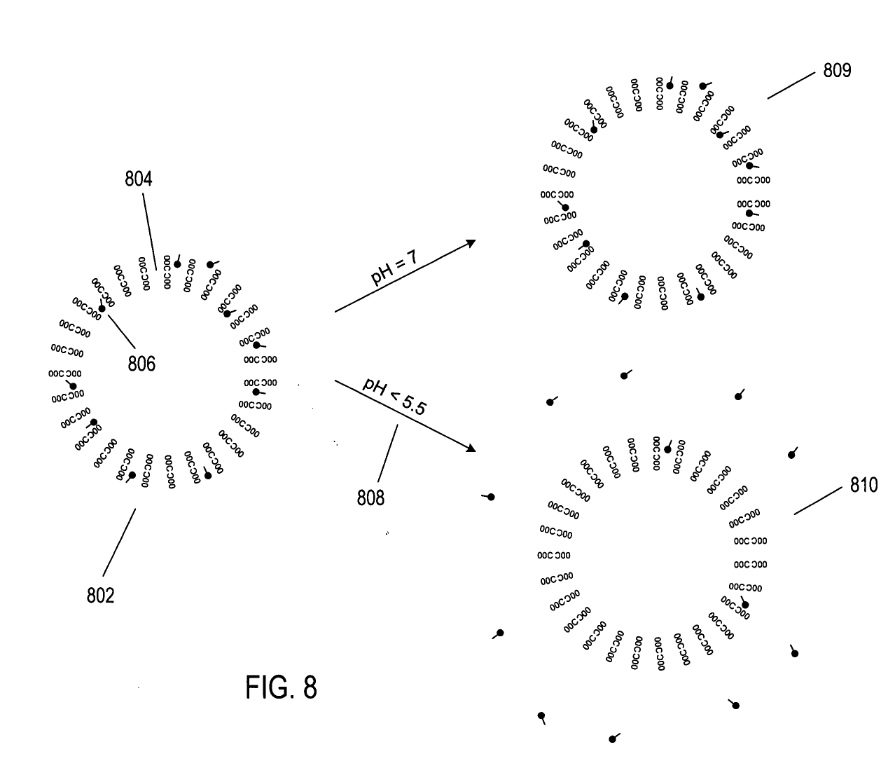

FIG. 8 illustrates the drug-incorporation and drug-release characteristics of lipid-drug complexes to which the present application is directed. In FIG. 8, a stable lipid-drug complex 802 is schematically shown as an annular lipid bilayer 804 with hydrophilic inner and outer surfaces and one or more hydrophobic domains within which a drug, represented by symbols such as symbol 806, is stably incorporated. In general, the lipid-drug complexes are generated by dissolving both the amphiphilic lipid-bilayer components and the drug in an organic solvent and then rehydrating the co-dissolved amphiphilic lipid-bilayer components and the drug to provide for complete or nearly complete incorporation of drug at neutral pH into the lipid-drug complex that forms when the co-dissolved lipid-bilayer components and drug are reintroduced into an aqueous medium. As discussed above, the drug is generally soluble or has low solubility (less than 10⁻⁴ moles/liter) in aqueous solutions at neutral pH and is lipophilic at neutral pH, leading to complete or nearly complete incorporation of the drug into stable lipid-drug complexes prepared by co-dissolving the drug and lipids in an organic solvent and then rehydrating the associated lipid and drug. This nearly complete incorporation of drugs having low solubility at neutral pH into the hydrophobic domain or domains of the lipid-drug complexes is generally relatively independent of the amphiphilic-component-complex composition, including whether or not additional components, such as cholesterol, are present. Furthermore, because the drug is tightly associated with the hydrophobic domain or domains of the amphiphilic-component complex, the complex need not be one or various a spherical or elliptical closed lipid-bilayer complexes, such as liposomes, which are characterized by having an interior aqueous volume fully enclosed by a lipid bilayer and therefore distinct and separate from the external aqueous media in which liposomes are suspended. As a result, formation of the lipid-drug-complexes to which the present application is directed does not involve or depend on creation of pH, salt, or charge gradients across a lipid bilayer in order to transport a drug from the external aqueous medium into the inner aqueous volume within a liposome or other fully enclosed structure. As a result, the drug is not sequestered, and does not precipitate, within the inner aqueous medium and is therefore released with much higher efficiency and under milder conditions than drugs encapsulated within liposomes.

As shown in FIG. 8, following generation of stable lipid-drug complexes in which the drug is incorporated within the hydrophobic domain or domains of the lipid complex 802, the stable lipid-drug complex retains all or nearly all of the incorporated drug when the pH is maintained within a range of pH's near the neutral pH 7.0 (809 in FIG. 8). However, as shown in FIG. 8 by arrow 808 and lipid-drug complex 810, when the pH is lowered below pH 5.5 808, the drug dissociates from the lipid-drug complex into the aqueous medium. At a pH below 5.0, nearly all of the previously incorporated drug dissociates from the lipid-drug complex. Drug dissociation does not depend on rupturing the lipid bilayer of a liposome or other closed structure, establishing reverse chemical and/or electrical gradients across a lipid bilayer, or resolublizing precipitated drug. Therefore, the dissociation of drug from the lipid-drug complexes to which the present application is directed proceeds with significantly greater efficiency under generally less severe conditions than dissociation of drug from the inner aqueous volume within closed lipid structures, such as liposomes.

TABLE 6
provided below, lists characteristics of lipid-indinavir-complex
formation and dissociation:

| | | | | pH sensitivity^{f} |
|---|---|---|---|---|
| Lipid-indinavir | Tm^{c} | Particle size^{d} | Degree of | (50% Indinavir |
| composition^{b} | (° C.) | (nm) | association^{e} | release) |
| EPC:CHOL | <4 | 35-50 | 100% | 5.5 |
| (7:3 m/m) | | | | |
| DSPC:DSPG | 55 | 62-66 | 99% | 5.2 |
| (8:2 m/m) | | | | |
| DSPC:mPEG-DSPE | 55 | 100-120 | 100% | 5.25 |
| (8:2 m/m) | | | | |
| DPPC:DPPG | 41 | 45-60 | 98% | 5.25 |
| (95:5 m/m) | | | | |

^{a}Indinavir-lipid complexes were prepared with the listed lipid compositions. The final lipid to indinavir mole ratio was fixed at 3:1 and drug indinavir was dissolved in organic phase with lipid. They are dried under nitrogen and reduced pressure. The drug associated nanoparticle is formed by addition of buffer at neutral pH and the size is reduced by sonication. The % drug association was determined. The pH sensitivity was measured by incubation of the complexes in varying pH conditions to determine the mid-point of pH when 50% of drug release occurred.
^{b}EPC: egg phosphatidyl choline, CHOL: cholesterol, DPPC: dipalmitoyl phosphatidyl choline, DPPG: dipalmitoyl phosphatidyl glycerol, DSPC: distearoyl phosphatidyl choline, DSPG: distearoyl phosphatidyl glycerol, mPEG-DSPE: methyl polyethylene glycol (MW: 2000)-phosphatidyl ethanolamine.
^{c}Tm, estimated gel-to-liquid lipid phase transition temperature, which is expressed in centigrade.
^{d}Particle size of the lipid-indinavir complexes was estimated by photon correlation spectroscopy.
^{e}Degree of drug association was determined by equilibrium dialysis to separate free and lipid associated indinavir. Batch to batch variation was less than 5%.
^{f}pH dependent release of drug was measured by incubating the complexes in pH 3-9, at 37° C. and data were presented as pH value at which 50% drug release occurs. Batch to batch and run to run variation was less than 10%.

To determine whether lipid composition influences the ability of indinavir to incorporate into lipid-drug nanoparticles, particles composed of lipids, exhibiting different phase transition temperatures (<4-56 C) and surface net charge were prepared. The lipid-to-drug ratio was kept constant at 3:1 for these studies. For overall negative net charge, we included two phosphatidyl glycerols (“PG”) with two different chain lengths of the fatty acyl chains [disteroyl-PG (DSPG) and dipalmitoyl-PG (DPPG)]. The results showed that, regardless of lipid composition, degree of saturation, and fatty acyl chain length, almost all indinavir was incorporated into lipid-drug nanoparticles at neutral pH (98-100% association, Table 6). Particle-size analysis with photon-correlation spectrometry indicated that lipid-drug nanoparticles composed of PEG conjugated lipid (DSPC:mPEG-DSPE) exhibited larger nanoparticles (100-120 nm diameter), compared to others (35-66 nm diameter). The pH-dependent dissociation of indinavir from lipid-drug particles was next determined. It was found that 50% of indinavir dissociation from the lipid-drug complexes occurred in the range of pH 5.2-5.5, regardless of lipid composition. Specifically, for EPC:CHOL, DSPC:DSPG, DSPC:mPEG-DSPE, DPPC:DSPG, half-maximum release was recorded at pH 5.5, 5.2, 5.25, and 5.25, respectively (Table 6).

Collectively, the data provided in Table 6 indicate that, regardless of lipid composition or particle size, the lipid-drug complexes incorporate indinavir with high efficiency (>95%). All lipid-drug compositions tested release 50% of indinavir from nanoparticles at pH 5.2-5.5.

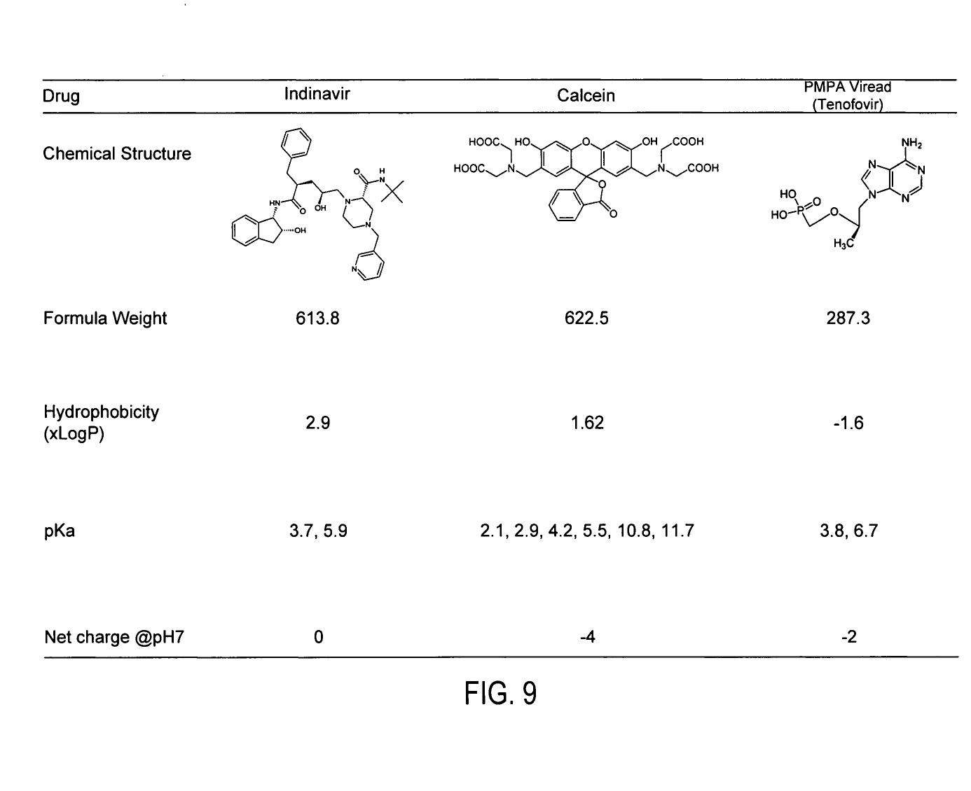

FIG. 9 provides a table showing characteristics of the drugs indinavir and tenofovir and the dye calcein. The drug indinavir and the dye calcein both incorporate, completely or nearly completely, within stable lipid-drug or lipid-dye complexes to which the present application is directed. Tenofovir, which is soluble in water and is hydrophilic, rather than hydrophobic, as illustrated in the table provided in FIG. 9, incorporates with quite low efficiency into the lipid-drug complexes.

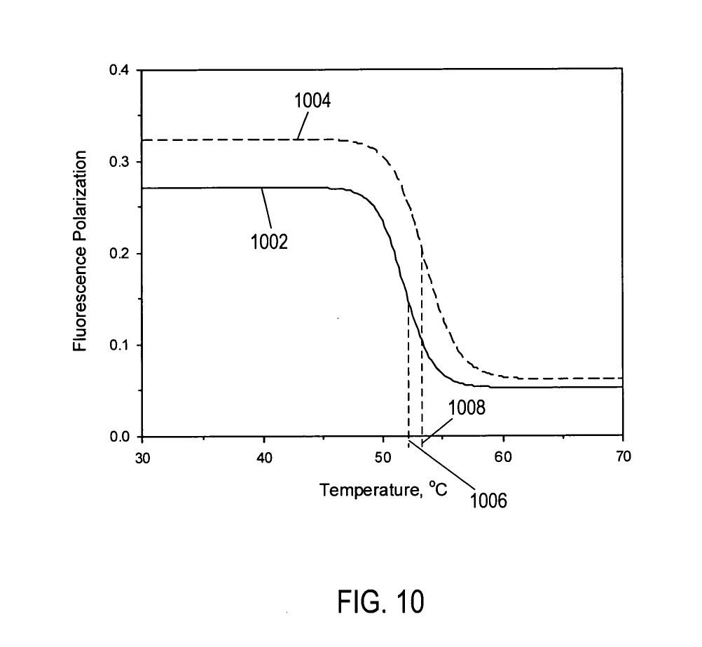

FIG. 10 provides a plot of fluorescence polarization versus temperature, as measured by using a diphenylhexatriene (“DPH”) fluorescence membrane probe, for indinavir incorporated within the hydrophobic domain of a phospholipid bilayer. DPH inserted within the hydrophobic domain of the phospholipid bilayer has been used to investigate membrane fluidity. When indinavir incorporates into the lipid bilayer, indinavir affects the lipid packing, detectable as changes in DPH anisotropy. Therefore, fluorescence anisotropy of DPH was monitored in a DSPC:mPEG lipid membrane with or without indinavir as a function of temperature.

As shown in FIG. 10, incorporation of indinavir into lipid bilayers decreased the polarization of DPH in lipid nanoparticles (curve 1002) compared to that of control lipid nanoparticles (curve 1004). These data are consistent with the insertion of indinavir in the lipid membrane. In addition, nanoparticles without indinavir incorporated within phospholipid bilayers showed a phase transition temperature (Tₘ) of 53.4 C (1008 in FIG. 10), which is similar to those reported elsewhere (Tₘ of DSPC, 54-56C), while nanoparticles with indinavir had a lower Tₘ of 51.9 C 1006 (1002 in FIG. 10). Collectively, these data indicate that indinavir incorporates within the lipid bilayer, increases the membrane fluidity in the gel phase, and reduces the phase transition temperature by 1.5 C.

Collectively these data provide direct evidence that drugs, such as indinvair, ritonavir, lopinavir, nenelfinavir and other protease inhibitors and drugs with titratable side chains that are lipid soluble in neutral pH, may be incorporated at incorporation efficiencies of nearly 100% into lipid-drug complexes. Such incorporation is independent of lipid composition, salt or pH gradients, and other severe conditions employed to release drugs enclosed within liposomes. Because many drugs, including HIV protease inhibitors, have stringent active-site binding requirements and in vivo pharmacokinetic profiles, most of the clinically-used drugs exhibit a profile with similar hydrophobicity and pH-dependent solubility as those of indinavir.

The drugs indinavir, saquinavir, and nelfinavir, all protease inhibitors, are seen to incorporate nearly completely into stable lipid-drug complexes to which the present application is directed. These three drugs are insoluble or have low solubility at neutral pH, and thus fit the profile of drugs and other compounds that can be incorporated by co-dissolving the drugs and other compounds with amphiphilic-complex components and then reintroducing the lipid-associated drugs and other compounds into aqueous medium to form stable lipid-drug and lipid-compound complexes at neutral pH, as discussed above. By contrast, the drug tenofovir, which is quite soluble in water at neutral pH, incorporates with much lower efficiency into the lipid-drug complex created by the above-discussed method. The efficiency and completeness of drug incorporation for indinavir, saquinavir, and nelfinavir into lipid-drug complexes to which the present application is directed and the efficiency and completeness of drug release at pH's lower than 5.5 fully distinguish these lipid-drug complexes from previously described liposome-encapsulated-drug pharmaceuticals. The latter incorporate drug with markedly less efficiency and release the incorporated drugs with markedly lower efficiencies. The liposome-encapsulated drugs are generally prepared by forcing drugs through the lipid bilayer using chemical and/or electrical gradients, a process with much lower efficiency than co-dissolving amphiphilic drugs with low aqueous solubility and amphiphilic-complex components in organic solvents. Drugs with low solubility in water, such as indinavir, that are precipitated within the inner aqueous medium of liposomes are are not released readily from the liposomes, but instead are released only under relatively severe conditions by establishing reverse chemical or electrical gradients to force the drug back across the lipid bilayer, rupturing the lipid bilayer, or changing the inner aqueous medium environment to resolublize precipitated drug.

Although the present invention has been described in terms of particular embodiments, it is not intended that the application be limited to these embodiments. Modifications within the spirit of the present invention will be apparent to those skilled in the art. For example, as discussed above, any of many different procedures, using any of various is lipophilic drugs with low solubility at neutral pH and many different lipid compositions, can be employed to create stable lipid-drug complexes that release a large fraction of the lipid-associated drug at a pH of 5.5 or lower.

It is appreciated that the previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

1. A lipid-drug complex comprising:
one or more types of amphiphilic lipid components that aggregate in aqueous media to produce stable lipid complexes that include one or more interior hydrophobic domains and external hydrophilic surfaces; and
at least one type of drug that has a solubility of less than 1×10⁻⁴ moles/liter in aqueous media at neutral pH and that incorporates within the one or more hydrophobic domains of the stable lipid complexes at an efficiency of at least 95% when the at least one drug and to amphiphilic lipid components are co-dissolved in a solvent and subsequently dissolved in an aqueous medium with an approximately neutral pH.

2. The lipid-drug complex of claim 1 wherein at least 50% of the at least one type of drug incorporated within the lipid-drug complex dissociates from the lipid-drug complex when the pH of the aqueous medium is lowered from neutral pH to pH 5.2.

3. The lipid-drug complex of claim 1 wherein the drug-to-lipid molar ratio is between 3:1 and 10:1.

4. The lipid-drug complex of claim 1 wherein the at least one type of drug is selected from among:
a protease-inhibiting anti-HIV drug;
a cancer-inhibiting drug;
an antifungal drug;
an antibacterial drug;
an immunomodulatory drug;
an analgesic drug;
an cardiovascular drug; and
an neurological drug.

5. The lipid-drug complex of claim 1 wherein the at least one type of drug is selected from among:
indinavir;
ritonavir:
lopinavir;
saquinavir; and
nelfinavir.

6. The lipid-drug complex of claim 1 wherein the one or types of amphiphilic lipid components are selected from among:
phosphatidylcholine;
phosphatidic acid;
lysophosphatidylcholine;
phosphatidylserine;
phosphatidylethanolamine;
sphingolipids;
phosphatidyglycerol;
spingomyelin;
cardiolipin;
glycolipids;
gangliosides;
cerebrosides;
long-chained aliphatic amines;
long-chained carboxylic acid;
long-chained sulfates;
long-chained phosphates;
long-chained alcohols;
egg phosphatidylcholine;
dilauryloylphosphatidylcholine;
dimyristoylphosphatidylcholine;
dipalmitoylphosphatidylcholine;
distearoylphosphatidylcholine;
1-myristoyl-2-palmitoylphosphatidylcholine;
1-palmitoyl-2-myristoyl phosphatidylcholine;
1-palmitoyl-2-stearoyl phosphatidylcholine;
1-stearoyl-2-palmitoyl phosphatidylcholine;
dioleoylphosphatidylycholine;
dilauryloylphosphatidylglycerol;
dimyristoylphosphatidylglycerol;
dipalmitoylphosphatidylglycerol;
distearoylphosphatidylglycerol;
distearoyl sphingomyelin;
distearoylphophatidylethanolamine;
dioleoylphosphatidylglycerol;
dimyristoyl phosphatidic acid;
dipalmitoyl phosphatidic acid;
dimyristoyl phosphatidylethanolamine;
dipalmitoyl phosphatidylethanolamine;
dimyristoyl phosphatidylserine;
dipalmitoyl phosphatidylserine;
brain phosphatidylserine;
brain sphingomyelin; and
dipalmitoyl sphingomyelin.

7. A method of administering a drug to a patient in need thereof by subcutaneously administering a lipid drug complex comprising:
one or more types of amphiphilic lipid components that aggregate in aqueous media to produce stable lipid complexes that include one or more interior hydrophobic domains and external hydrophilic surfaces; and
at least one type of drug that has a solubility of less than 1×10⁻⁴ moles/liter in aqueous media at neutral pH and that incorporates within the one or more hydrophobic domains of the stable lipid complexes at an efficiency of at least 95% when the at least one drug and amphiphilic lipid components are co-dissolved in a solvent and subsequently dissolved in an aqueous medium with an approximately neutral pH.

8. The method of claim 8 where the lipid-drug complex is subcutaneously administered to lymphatic tissue or cells.

9. A method of preparing lipid-drug complexes comprising:
co-dissolving one or more types of amphiphilic lipid components that aggregate in aqueous media and at least one type of drug that has a solubility less than 1×10⁻⁴ moles/liter in aqueous media at neutral pH in a solvent;
evaporating essentially all of the co-solvent;
re-suspending the one or more types of amphiphilic lipid components and the at least one type of drug in an aqueous medium with an approximately neutral pH where the at least one type of drug is incorporated at an efficiency of at least 95% homogenizing the re-suspended solution.
